SEQUENCE LISTING (10 sequences)

SEQ ID NO: 1 - RNA, 20 nt, synthetic construct
  source 1..20: mol_type = other RNA
cggcccttgtctgcaaacag

SEQ ID NO: 2 - RNA, 20 nt, synthetic construct
  source 1..20: mol_type = other RNA
aaagaacaaatggaacgcga

SEQ ID NO: 3 - DNA, 20 bp, synthetic construct
  source 1..20: mol_type = other DNA
cctgtcatccccgtctacac

SEQ ID NO: 4 - DNA, 19 bp, synthetic construct
  source 1..19: mol_type = other DNA
cacatggagtccgccgtaa

SEQ ID NO: 5 - DNA, 21 bp, synthetic construct
  source 1..21: mol_type = other DNA
cgcatcaacagcagcatcgag

SEQ ID NO: 6 - DNA, 20 bp, synthetic construct
  source 1..20: mol_type = other DNA
gacgaaggctttgctgtgct

SEQ ID NO: 7 - DNA, 19 bp, synthetic construct
  source 1..19: mol_type = other DNA
ggctcctggcaaaaggtca

SEQ ID NO: 8 - DNA, 20 bp, synthetic construct
  source 1..20: mol_type = other DNA
ctgcgtagttgtgctgatgt

SEQ ID NO: 9 - DNA, 22 bp, synthetic construct
  source 1..22: mol_type = other DNA
ttgccgacaggatgcagaagga

SEQ ID NO: 10 - DNA, 22 bp, synthetic construct
  source 1..22: mol_type = other DNA
aggtggacagcgaggccaggat

(19) United States
(12) Patent Application Publication
(10) Pub. No.: US 2026/0023068 A1
(43) Pub. Date: Jan. 22, 2026

(54) USE OF A FBXO42 SPECIFIC INHIBITOR IN TREATING NOTCH SIGNALING-DEPENDENT DISEASE

(71) Applicant: WESTLAKE UNIVERSITY, Zhejiang (CN)

(72) Inventor: Xu LI, Zhejiang (CN)

(73) Assignee: WESTLAKE UNIVERSITY, Zhejiang (CN)

(21) Appl. No.: 18/993,461

(22) PCT Filed: Jul. 10, 2023
(86) PCT No.: PCT/CN2023/106618

§ 371 Date: Jan. 10, 2025

(30) Foreign Application Priority Data
Jul. 11, 2022 (WO) PCT/CN2022/104922

(51) Int. Cl. G01N 33/50

(52) U.S. Cl. CPC G01N 33/5011; G01N 2500/02

(57) ABSTRACT

The present invention refers to a method for treating Notch signaling-dependent disease in the subject with a FBXO42 specific inhibitor. The Notch signaling-dependent disease is selected from leukemia. Also provided is a method for screening a drug treating Notch signaling-dependent disease using FBXO42 as a target.

### CROSS REFERENCE TO RELATED APPLICATIONS

This application claims the benefit of, and priority to, PCT patent application serial number PCT/CN2022/104922, filed Jul. 11, 2022, which is hereby incorporated by reference herein in its entirety.

### TECHNICAL FIELD

The present disclosure generally relates to a method for the down-regulation of FBXO42 using related inhibitors to treat Notch signaling-dependent disease.

### BACKGROUND

The Notch signaling pathway is one of the most dysregulated pathways in cancer.

Alterations include activating mutations and amplification of Notch pathway activity, leading to the progression of cancers, especially T-cell acute lymphoblastic leukemia/lymphoma (T-ALL) (A. P. Weng et al., Activating mutations of NOTCH1 in human T cell acute lymphoblastic leukemia. Science 306, 269-271 (2004)), chronic lymphocytic leukemia (C-ALL) (X. S. Puente et al., Non-coding recurrent mutations in chronic lymphocytic leukaemia. Nature 526, 519-524 (2015)), diffuse large B-cell lymphoma (DLBCL) (K. Karube et al., Integrating genomic alterations in diffuse large B-cell lymphoma identifies new relevant pathways and potential therapeutic targets. Leukemia 32, 675-684 (2018)), head and neck squamous cell carcinoma (HNSCC) (N. Agrawal et al., Exome sequencing of head and neck squamous cell carcinoma reveals inactivating mutations in NOTCH1. Science 333, 1154-1157 (2011); N. Stransky et al., The mutational landscape of head and neck squamous cell carcinoma. Science 333, 1157-1160 (2011)) and breast cancers (D. R. Robinson et al., Functionally recurrent rearrangements of the MAST kinase and Notch gene families in breast cancer. NatMed 17, 1646-1651 (2011); S. Stylianou, R. B. Clarke, K. Brennan, Aberrant activation of notch signaling in human breast cancer. Cancer Res 66, 1517-1525 (2006)). Therapeutic strategies to modulate Notch pathway function include chemical and immunological targeting of Notch receptors, delta ligands, and γ-secretases (Y. Wu et al., Therapeutic antibody targeting of individual Notch receptors. Nature 464, 1052-1057 (2010); M. Lopez-Guerra et al., Specific NOTCH1 antibody targets DLL4-induced proliferation, migration, and angiogenesis in NOTCH1-mutated CLL cells. Oncogene 39, 1185-1197 (2020); P. J. Real et al., Gamma-secretase inhibitors reverse glucocorticoid resistance in T cell acute lymphoblastic leukemia. Nat Med 15, 50-58 (2009)). Although the Notch pathway has been studied in past decades, the use of pharmacological compounds targeting Notch activity in clinical settings is still insufficient, especially in Notch-activated T-cell leukemia. To date, γ-secretase inhibitors have been the most extensively explored potential anticancer agents in these contexts. However, due to the side effects induced by γ-secretase inhibitors in clinical settings (J. H. van Es et al., Notch gamma-secretase inhibition turns proliferative cells in intestinal crypts and adenomas into goblet cells. Nature 435, 959-963 (2005)) and because mutant Notch does not require γ-secretase cleavage to be activated (A. P. Weng et al., Activating mutations of NOTCH1 in human T cell acute lymphoblastic leukemia. Science 306, 269-271 (2004)), the need to develop new strategies by identifying novel molecular targets, especially components downstream of Notch activation, remains urgent.

Recombination signal-binding protein of immunoglobulin kappa J region (RBPJ), a transcription factor in the Notch signaling pathway, plays a dual role in regulating Notch signaling. In the absence of the Notch intracellular domain (NICD), RBPJ acts as a transcriptional repressor of Notch target genes, exerting its effect by interacting with corepressor complexes such as histone deacetylases (H. Y. Kao et al., A histone deacetylase corepressor complex regulates the Notch signal transduction pathway. Genes Dev 12, 2269-2277 (1998)), lysine-specific histone demethylase 1A (P. Mulligan et al., A SIRT1-LSD1 corepressor complex regulates Notch target gene expression and development. Mol Cell 42, 689-699 (2011)) and lethal(3) malignant brain tumor-like protein 3 (L3MBTL3) (T. Xu et al., RBPJ/CBF1 interacts with L3MBTL3 MBT1 to promote repression of Notch signaling via histone demethylase KDM1A LSD1. EMBO J 36, 3232-3249 (2017)). Upon Notch activation, RBPJ associates with the NICD and masterminds (MAMLs) to form a ternary complex, recruiting coactivators such as the histone acetyltransferases p300 and GCN5 and triggering the transcription of Notch target genes (H. Kurooka, T. Honjo, Functional interaction between the mouse notch1 intracellular region and histone acetyltransferases PCAF and GCN5. J Biol Chem 275, 17211-17220 (2000)).

Despite the progress made in delineating the molecular structures of the transcriptional complex in the past decade, the mechanism of RBPJ function switching remains unclear, making it difficult to target the Notch transcription step. Depletion of RBPJ leads to Notch signaling inactivation in certain cellular contexts (E. Vasyutina et al., RBP-J (Rbpsuh) is essential to maintain muscle progenitor cells and to generate satellite cells. Proc Natl Acad Sci USA 104, 4443-4448 (2007)) and to Notch signaling activation in other contexts (I. Kulic et al., Loss of the Notch effector RBPJ promotes tumorigenesis. J Exp Med 212, 37-52 (2015)), making pharmacologically targeting RBPJ in Notch-related cancers very risky.

RBPJ/NICD dimerization is suspected to be a stabilizing event enabling RBPJ binding (Y. Nam, P. Sliz, W. S. Pear, J. C. Aster, S. C. Blacklow, Cooperative assembly of higher-order Notch complexes functions as a switch to induce transcription. Proc Natl Acad Sci USA 104, 2103-2108 (2007); M. R. Hass et al., SpDamID: Marking DNA Bound by Protein Complexes Identifies Notch-Dimer Responsive Enhancers. Mol Cell 59, 685-697 (2015); H. Liu et al., Notch dimerization is required for leukemogenesis and T-cell development. Genes Dev 24, 2395-2407 (2010)); however, the DNA-binding affinity of RBPJ is surprisingly low (Kd˜1 μM) (R. Torella et al., A combination of computational and experimental approaches identifies DNA sequence constraints associated with target site binding specificity of the transcription factor CSL. Nucleic Acids Res 42, 10550-10563 (2014)), and the binding of NICD to RBPJ does not influence RBPJ binding affinity for DNA (R. Torella et al., A combination of computational and experimental approaches identifies DNA sequence constraints associated with target site binding specificity of the transcription factor CSL. Nucleic Acids Res 42, 10550-10563 (2014)). It remains to be elucidated whether the plasticity of DNA binding by RBPJ is due to cofactors that can sense chromatin structure (R. Liejke et al., Histone demethylase KDM5A is an integral part of the core Notch-RBP-J repressor complex. Genes Dev 24, 590-601 (2010); F. Oswald et al., A phospho-dependent mechanism involving NCoR and KMT2D controls a permissive chromatin state at Notch target genes. Nucleic Acids Res 44, 4703-4720 (2016)) or whether RBPJ cooperates with other DNA-binding proteins to prolong its association with chromatin (A. S. Geimer Le Lay et al., The tumor suppressor Ikaros shapes the repertoire of notch target genes in T cells. Sci Signal 7, ra28 (2014); S. Chari, S. Winandy, Ikaros regulates Notch target gene expression in developing thymocytes. J Immunol 181, 6265-6274 (2008)). In any case, the role of RBPJ is controversial and context-dependent, and the mechanism by which the RBPJ transcriptional switch is fine-tuned remains to be elucidated.

In this study, we established a detailed RBPJ interactome via tandem-affinity purification coupled with mass spectrometry (TAP-MS) and explored the potential regulators critical for RBPJ transcriptional activities. We found that FBXO42 physically and functionally interacted with RBPJ, mediating its K63-linked polyubiquitination and contributing to its binding to chromatin, the conformation of which was subsequently opened, as well as Notch signaling activation. Both genetic knockout (KO) of FBXO42 and pharmacological inhibition of FBXO42 action alleviated leukemia progression in vivo, exhibiting therapeutic value in Notch-associated disease.

### SUMMARY OF INVENTION

The present disclosure provides a novel method of treating Notch signaling-dependent disease. In certain embodiments, the present disclosure provides a method for treating Notch signaling-dependent disease by using a FBXO42 specific inhibitor, which may be a polypeptide antagonist specifically against FBXO42, a polynucleotide specific to FBXO42, or a small molecule compound inhibitor specific to FBXO42. Preferably, the Notch signaling-dependent disease include activating mutations and/or amplification of Notch gene and/or Notch pathway activity, preferably, the Notch signaling-dependent disease is selected from leukemia, myeloma, lymphoma, breast cancer, liver cancer, head and neck squamous cell carcinoma (HNSCC), lung cancer and other cancers carrying the activating mutations and/or amplification of Notch gene and/or Notch pathway activity.

In one aspect, the disclosure provides a FBXO42 specific inhibitor for use in treating Notch signaling-dependent disease. The FBXO42 inhibitor is selected from a polypeptide antagonist specifically against FBXO42, a polynucleotide specific to FBXO42, or a small molecule compound inhibitor specific to FBXO42. Preferably, the Notch signaling-dependent disease include activating mutations and/or amplification of Notch gene and/or Notch pathway activity, preferably, the Notch signaling-dependent disease is selected from leukemia, myeloma, lymphoma, breast cancer, liver cancer, head and neck squamous cell carcinoma (HNSCC), lung cancer and other cancers carrying the activating mutations and/or amplification of Notch gene and/or Notch pathway activity.

In one aspect, the invention provides use of a FBXO42 specific inhibitor in preparation of medicine for treating Notch signaling-dependent disease. The FBXO42 inhibitor is a polypeptide antagonist specifically against FBXO42, a polynucleotide specific to FBXO42, or a small molecule compound inhibitor specific to FBXO42. Preferably, the Notch signaling-dependent disease include activating mutations and/or amplification of Notch gene and/or Notch pathway activity, preferably, the Notch signaling-dependent disease is selected from leukemia, myeloma, lymphoma, breast cancer, liver cancer, head and neck squamous cell carcinoma (HNSCC), lung cancer and other cancers carrying the activating mutations and/or amplification of Notch gene and/or Notch pathway activity.

In one embodiment, the small molecule compound is a small molecule inhibitor targeting NEDD8-activating enzyme (NAE).

In one embodiment, the polypeptide antagonist is an antibody against FBXO42.

In one embodiment, the polynucleotide is selected from siRNA, shRNA, guide RNA, miRNA, and ASO.

In certain embodiments, the polynucleotide specific to FBXO42 comprises a nucleotide sequence of SEQ ID NO: 1, a nucleotide sequence with at least 70%, 80%, 85%, 90%, 95%, 99%, or more identity to SEQ ID NO:1, or an amino acid sequence with addition, deletion and/or substitution of one or more amino acids compared with SEQ ID NO: 1, and the polynucleotide specific to FBXO42 can prevent ligands such as from its binding.

In one embodiment, the Notch signaling-dependent disease is selected from leukemia e.g., T-acute lymphoblastic leukemia or Chronic lymphocytic leukemia, myeloma e.g. Multiple myeloma, lymphoma e.g. Hodgkin lymphoma, Burkitt lymphoma, Diffuse large B-cell lymphoma, Mantle cell lymphoma, Splenic marginal zone lymphoma, Follicular lymphoma, breast cancer, liver cancer, lung cancer, head and neck squamous cell carcinoma (HNSCC), and lung adenocarcinoma cells. The leukemia is T-acute lymphoblastic leukemia or Chronic lymphocytic leukemia. In one specific embodiment, the disease is any type of leukemia. These diseases are with Notch signaling activation or upregulation, preferably, the Notch signaling-dependent disease comprises Notch related mutations, more preferably, Notch related mutations comprise Notch1, Notch2 and/or Notch3 mutations.

In one embodiment, the subject is non-human mammal or human.

In other aspect, the invention provides a method of screening medicines for treating Notch signaling-dependent disease using FBXO42 as the target, the method comprising: observing the effect of candidate medicine on the expression or activity level of FBXO42, if the candidate medicine can inhibit expression or activity level of FBXO42, then it indicates that the candidate medicine is a potential medicine for treating Notch signaling-dependent disease. In one embodiment, the Notch signaling-dependent disease is selected from leukemia e.g., T-acute lymphoblastic leukemia or Chronic lymphocytic leukemia, myeloma e.g. Multiple myeloma, lymphoma e.g. Hodgkin lymphoma, Burkitt lymphoma, Diffuse large B-cell lymphoma, Mantle cell lymphoma, Splenic marginal zone lymphoma, Follicular lymphoma, breast cancer, liver cancer, lung cancer, head and neck squamous cell carcinoma (HNSCC), and lung adenocarcinoma cells. The leukemia is T-acute lymphoblastic leukemia or Chronic lymphocytic leukemia. In one specific embodiment, the disease is any type of leukemia. These diseases are with Notch signaling activation or upregulation, preferably, the Notch signaling-dependent disease comprises Notch related mutations, more preferably, Notch related mutations comprise Notch1, Notch2 and/or Notch3 mutations.

### BRIEF DESCRIPTION OF DRAWINGS

FIG. 1 shows FBXO42 is a novel interactor of RBPJ and a positive regulator of the Notch pathway. (A) Interaction network of RBPJ. HCIPs were grouped based on their cellular functions as indicated by GO analysis and a literature search. Ubiquitination (Ub)/ubiquitin-like (UbL)-related proteins, proteins that are potential positive and negative regulators are indicated by different colored dots. Signaling pathways and proteins reported to be involved in Notch pathway regulation are indicated by different colored text. NER, nuclear excision repair; BER, base excision repair; Th1/Th2, T Helper 1/2 cells. (B and C) The expression of L3MBTL3 (B) and Notch target genes (C) was evaluated in WT and L3MBTL3-KO HEK293T cells. (D and E) The expression of FBXO42 (D) and Notch target genes (E) was evaluated in WT and FBXO42-KO HEK293T cells. (F and G) GFP reporter (F) and luciferase assays (G) were performed to evaluate Notch signaling activities in WT and FBXO42-KO HEK293T cells. (H-J) The expression level of FBXO42 in Notch related cancers as indicated was analyzed using TCGA datasets. (K and L) The expression level of FBXO42 in various cancer cell lines (K) and T-ALL cell lines (L) derived from the Cancer Cell Line Encyclopedia (https://sites.broadinstitute.org/ccle). (M and N) The expression correlation of FBXO42 and Notch pathway target genes in DLBCL and AML patients was analyzed using UALCAN database (https://software.broadinstitute.org/morpheusualcan.path.uab.edu/home). (B-G, n=3) Quantitative data are presented as mean±SEM from three independent experiments. P values were calculated using two-tailed Student's t-tests. *P<0.05, ***P<0.001, ****P<0.0001, NS, not significant.

FIG. 2 shows enrichment analysis and validation of the RBPJ interactors, and FBXO42 correlation with Notch pathway. (A-C) Biological process (A), cellular component (B) and KEGG enrichment (C) analyses of the genes encoding RBPJ interactors. The x-axis represents gene ratio=count/set size. The size of the dots represents the number of genes associated with a GO term, and the color of the dots represents the adjusted P-values. (D) Top RBPJ interactors identified in this study were selected for use in a co-immunoprecipitation (co-IP) assay to validate their interactions with RBPJ. HEK293T cells were cotransfected with Myc-tagged constructs and cSFB-tagged RBPJ. The cell lysates were incubated with S beads. Five percent lysate was used as the input control. Blots with antibodies recognizing the FLAG- and Myc-epitope tags and Actin are shown. (E and F) The expression level of FBXO42 in HNSCC (E) and ALL (F) patients from TCGA datasets. (G) Expression correlation between FBXO42 and Notch target genes in ALL patients using GEO datasets. P values were calculated using two-tailed Student's t-tests. *P<0.05, ****P<0.0001.

FIG. 3 shows FBXO42 directly interacts with RBPJ. (A) Interaction network of FBXO42. Top 50 interactors identified in TAP-MS was shown. (B) KEGG enrichment analysis of preys acquired in FBXO42 tandem-affinity purification (TAP)-MS. The x-axis represents gene ratio=count/set size. The size of the dots represents the number of genes associated with the GO term and the color of the dots represent the adjusted P-values. (C) Circos plot showing overlapping RBPJ and FBXO42 preys. Purple lines link the genes that shared by RBPJ and FBXO42. Blue lines link different genes enriched in the same ontology term. (D and E) HEK293T cell lysates were incubated with IgG control and antibodies against FBXO42 (D) or RBPJ (E). Five percent lysate was used as the input control. Blots with antibodies recognizing RBPJ, FBXO42 and Actin are shown. (F and G) HEK293T cells were cotransfected with Myc-tagged FBXO42 and cSFB-tagged RBPJ, as indicated. The cell lysates were incubated with S beads (F) or an anti-MYC antibody (G). Five percent lysate was used as the input control. Blots with antibodies recognizing the FLAG and MYC epitope tags and Actin are shown. (H) Coomassie blue staining of cSFB-tagged RBPJ and FLAG-FBXO42 proteins purified from 293F cells. (I) Biomolecular interaction kinetics curve showing the binding affinity between purified RBPJ and FBXO42 proteins. The RBPJ was used at 225 nM for binding to SA probe. The color indicates the concentration gradient of FBXO42 protein (19.5 nM-625 nM). (J and K) Schematics showing RBPJ (J) and FBXO42 (K) domain deletion mutants used in the domain mapping assays. (L and M) HEK293T cells were cotransfected with Myc-tagged FBXO42 and cSFB-tagged WT or mutant RBPJ (L) or cSFB-tagged RBPJ and Myc-tagged WT or mutant FBXO42 (M). (N) HEK293T cells were cotransfected with Myc-tagged FBXO42 Kelch domain and cSFB-tagged RBPJ NTD as indicated. (L-N) The cell lysates were incubated with S beads. Five percent lysate was used as the input control. Blots with antibodies recognizing the FLAG and MYC epitope tags and Actin are shown. (D-I, L-N, n=3).

FIG. 4 shows FBXO42 promotes RBPJ K63-linked polyubiquitination and positively regulates Notch signaling. (A) cSFB-RBPJ, hemagglutinin-ubiquitin (HA-Ub) and Myc-FBXO42 WT were cotransfected into HEK293T cells as indicated and treated with or without MG132 for 4 h. The cell lysates were incubated with S beads and analyzed by western blotting for RBPJ polyubiquitination detection. (B) Immunoprecipitated endogenous RBPJ products from WT and FBXO42 KO cells were immunoblotted for Ub. (C) cSFB-RBPJ, hemagglutinin-ubiquitin (HA-Ub) and Myc-FBXO42 WT or AF mutant were cotransfected into HEK293T cells. The cell lysate was collected and analyzed for RBPJ polyubiquitination. (D) HEK293T cells were transfected with cSFB-RBPJ, Myc-FBXO42 and HA-Ub with or without FLAG-NICD1 overexpression. The cell lysates were incubated with S beads and analyzed by western blotting for RBPJ polyubiquitination detection. (E) HEK293T cells were transfected with cSFB-RBPJ, Myc-FBXO42 or HA-Ub WT or mutants. The degree of RBPJ polyubiquitination was evaluated as described above. (F) The sequences of the predicted and identified peptides (the respective lysine residues are indicated) in mass spectrometry are shown. (G) Evaluating ubiquitination of five cSFB-RBPJ lysine mutants (K135R, K175R, K269R, K285R, and K315R). HEK293T cells were cotransfected with Myc-FBXO42, HA-Ub-K63along with cSFB-RBPJ WT or its lysine mutants as indicated. The cell lysates were incubated with S beads and analyzed by western blotting for RBPJ polyubiquitination detection. (H) Sequence alignment of RBPJ in different species showed the conservation of the K175 site. (I) HEK293T cells were transfected with cSFB-RBPJ WT or K175R mutant and treated with cycloheximide (CHX) for the indicated times, and the lysates were probed with an antibody against the FLAG epitope. (J) Quantitation of the results shown in (I). (K) HEK293T cells were transfected with various constructs as indicated, followed by treatment with DMSO or MLN4924. The cell lysates were analyzed for RBPJ ubiquitination. (L-N) qPCR analysis of the Notch target genes HES1 (L), HES5 (M), and c-MYC (N) in HEK293T cells overexpressing FBXO42, RBPJ WT or K175R mutant. (O) mRNA expression of Notch target genes in cells treated with MLN4924. (P) Efficiency of RBPJ short interfering RNAs (siRNAs) detected via western blotting. (Q) qPCR analysis of Notch target genes in RBPJ-knockdown cells. (R) HEK293T cells were transfected with si-Scramble or si-RBPJ and overexpressed with Myc-FBXO42 and then subjected to qPCR analysis of Notch target gene expression. (A-J, L-R, n=3). Quantitative data are presented as mean±SEM. P values were calculated using two-tailed Student's t-tests or ANOVA for multiple comparison. *P<0.05, **P<0.01, ***P<0.001, ##P<0.01 vs. FBXO42.

FIG. 5 shows FBXO42 mediated RBPJ polyubiquitination at lysine 175 site. (A) Endogenous RBPJ ubiquitination was measured under FBXO42 overexpression after immunoprecipitation with antibody against RBPJ and immunoblotted for Ub. Five percent lysate was used as the input control. (B) cSFB-RBPJ, hemagglutinin-ubiquitin (HA-Ub) and Myc-FBXO42 WT or AK mutant were cotransfected into HEK293T cells as indicated and treated with MG132 for 4 h. The cell lysates were incubated with S beads and analyzed by western blotting for RBPJ polyubiquitination detection. (C) Peptide sequence with ubiquitination modification identified in WT and FBXO42 KO cells. (D) HEK293T cells were cotransfected with Myc-FBXO42, HA-Ub WT along with cSFB-RBPJ WT or its lysine mutants as indicated. The cell lysates were incubated with S beads and analyzed by western blotting for RBPJ polyubiquitination detection. (E) Summary of identified peptides in global ubiquitination analysis upon MLN4924 treatment or FBXO42 depletion. (F) Percentage of ubiquitinated peptides among different groups. (G and H) Gene Ontology analysis of these proteins with differential ubiquitination modification. (I and J) Venn diagram showing the overlap between identified differential proteins with reported Notch interactors.

FIG. 6 shows FBXO42 regulates RBPJ chromatin association and transcriptional activity. (A) WT and FBXO42-KO HEK293T cells were harvested and subjected to subcellular fractionation. The nuclear, cytoplasm, chromatin and soluble fractions were isolated, and immunoblot analysis was performed. (B) HEK293T cells overexpressing RBPJ WT or K175R were harvested and the chromatin and soluble fractions were isolated, and then immunoblot analyses were performed. (C) WT and FBXO42 KO HEK293T cells were cotransfected with cSFB-RBPJ and Myc-p300. Cell lysates were collected, incubated with S-protein beads and blotted with antibodies against FLAG- or MYC-epitope tags. (D) WT and FBXO42-KO HEK293T cells were transfected with cSFB-RBPJ and used for S-protein bead pull-down assay, followed by immunoblotting with endogenous antibodies as indicated. (E) HEK293T cells expressing RBPJ WT or its K175R mutant were harvested and used for S-protein beads pull-down assay, followed by immunoblotting with endogenous antibodies as indicated. (F) WT and FBXO42-KO HEK293T cells were transfected with cSFB-RBPJ and Myc-FBXO42 WT or AF mutant. Then, the histone modifications were evaluated with western blotting using antibodies against H3K4m1 and H3K27ac. H3 and Actin served as the loading controls. (G) WT and FBXO42-KO HEK293T cells were collected for a CUT&Tag assay. Classical RBPJ motif identified in CUT&Tag assay was shown. (H) Representative images of RBPJ-binding sites in known Notch pathway target genes HES1, HES4 and MYC was shown. HSP90AA1 was used as the negative control. (I) Heatmap showing RBPJ CUT&Tag read densities of WT and FBXO42-KO HEK293T cells. (J) Signaling pathway enrichment of genes with differential RBPJ-binding affinities based on Gene Ontology annotation. The size of the dots represents the number of genes associated with the GO term and the color of the dots represent the adjusted P-values. (K) Heatmap showing CUT&Tag read densities of RBPJ WT and K175R mutant. (A-F, n=3).

FIG. 7 shows ubiquitinated RBPJ is more associated with chromatin and mediates its activity. (A) subcellular fractionation for detection of FBXO42 and its ubiquitin complex (SKP1, CUL1, RBX1). The lysate from nuclear, cytoplasm, soluble and chromatin fraction were used for immunoblotting as indicated. (B and C) RBPJ-ubiquitinated products are predominantly associated with the chromatin-enriched fraction. RBPJ ubiquitin assays were conducted in four subcellular fractions of chromatin containing WCL, cytosol, soluble and chromatin under FBXO42 depletion (B) or overexpression (C). (D-G) Representative motifs identified in CUT&Tag assay. (H) Luciferase reporter assay for detecting the effect of RBPJ K175R mutant on its transcription activity. (I-K) Endogenous RBPJ depleted JURKAT cells were overexpressed with RBPJ WT or K175R mutant and used for CUT&Tag assay. Representative images of CUT&Tag peak in Notch pathway target genes HES1, HES4 and MYC were shown. (A-C, n=3).

FIG. 8 shows FBXO42 mediates global chromatin remodeling in an RBPJ-dependent manner. (A) WT and FBXO42 KO cells were cotransfected with cSFB-RBPJ and Myc-tagged constructs encoding epigenetically modified proteins. Then, cell lysates were incubated with S-protein beads and blotted with antibodies against FLAG- or MYC-epitope tags. (B) WT and FBXO42-KO cells were cotransfected with cSFB-RBPJ and Myc-tagged constructs encoding SWI/SNF complex proteins. Then, the cells were harvested and analyzed as described in (A). (C) Heatmap showing the differential interaction between chromatin factors and RBPJ in WT and FBXO42 KO cells as detected by mass spectrometry. (D) Enrichment analysis of the differentially interacting proteins of heterochromatin components is shown on the basis of Gene Ontology annotation. (E) Immunofluorescence detection of HP1α foci in WT and FBXO42-KO cells. Scale bars, 10 μm. (F) HP1α foci number and percentage of HP1α foci area were calculated using Image J software. (G) WT and FBXO42-KO cells were digested with MNase for 3 min and chromatin relaxation was monitored by the release of nucleosomes. (H) Chromatin association of the SWI/SNF subunits SMARCA2, SMARCA4, and SMARCC2 in WT and FBXO42-KO cells was analyzed using western blotting after chromatin isolation. ORC2 served as the loading control. (I) WT and FBXO42-KO cells were digested with DNase for 3 min and followed with agarose gel electrophoresis analysis. (J) Chromatin from WT and FBXO42 KO cells was isolated, DNase I digested, and used as substrate for accessibility assay. (K) The heat map view for ATAC-seq signal intensity at transcription start sites in WT and FBXO42 KO JURKAT cells. (L) ATAC-seq peaks and H3K4m1, H3K4m3, H3K27ac ChIP-seq peaks as well as DNase-seq peaks downloaded from ENCODE database at MYC locus were analyzed. (A, B, E-J, n=3). Quantitative data are presented as mean±SEM. P values were calculated using two-tailed Student's t-tests. *P<0.05, **P<0.01.

FIG. 9 shows impact of FBXO42-RBPJ axis on chromatin remodeling. (A) HEK293T cells were transfected with cSFB-RBPJ WT or K175R and Myc-tagged constructs encoding epigenetically modified proteins. Then, cell lysates were incubated with S-protein beads and blotted with antibodies against FLAG- or MYC-epitope tags. (n=3). (B) Heatmap view of differential interactions between chromatin factors and RBPJ WT or its K175R mutant identified in mass spectrometry. (C-G) Volcano plot showing the differentially interacting proteins involved in tandem-affinity purification coupled with mass spectrometry (TAP-MS) of heterochromatin components. Proteins involved in chromatin remodeling are labeled. (H) ATAC-seq peaks and H3K4m1, H3K4m3, H3K27ac ChIP-seq peaks as well as DNase-seq peaks downloaded from ENCODE database at HES 1 locus were analyzed. (I and J) ChIP-qPCR for H3K4me3 (I) and H3K27ac (J) enrichment at Notch target genes promoter sites in JURKAT cells.

FIG. 10 shows FBXO42 plays an essential role in Notch signaling-dependent leukemogenesis. (A) Western blots with antibodies recognizing FBXO42 and Actin in various leukemia cell lines. PBSC represent the healthy hematopoietic cell. (B) The CRISPR/Cas9-mediated KO of FBXO42 in JURKAT and HSB2 cells was determined by western blotting. (C and D) The mRNA levels of the Notch target genes in WT and FBXO42-KO JURKAT (C) and HSB2 (D) cells were determined by qPCR. (E and F) The level of RBPJ in different fractions was determined for WT and FBXO42-KO JURKAT (E) and HSB2 (F) cells as described in FIG. 6A. (G) Chromatin association of the SWI/SNF subunits SMARCA2, SMARCA4, and SMARCC2 in WT and FBXO42-KO leukemia cells was analyzed using western blotting after chromatin isolation. (H-K) Invasion abilities of WT and FBXO42-KO JURKAT (H) and HSB2 (J) cells were measured using a three-dimensional culture system with Matrigel. Scale bars, 50 μm. The average diameter (I and K) of the spheres was determined. (L-O) Migration abilities of the WT and FBXO42-KO JURKAT (L) and HSB2 (N) cells were measured using a Transwell migration assay. Scale bars, 200 μm. The number of cells that migrated into the lower chamber was counted (M and O). (P-S) Anchorage-independent tumorigenesis abilities of the WT and FBXO42-KO JURKAT (P) and HSB2 (R) cells were measured with a soft agar colony formation assay. The number of colonies in P and R was counted, respectively (Q and S). (T-Y) FBXO42 functions rely on the presence of RBPJ. The KO efficiency of RBPJ in JURKAT (T) and HSB2 (W) cells was determined by western blotting. The mRNA levels of Notch target genes in WT and RBPJ-KO JURKAT (U) and HSB2 (X) cells were determined by qPCR. The mRNA levels of Notch target genes in RBPJ-KO JURKAT (V) and HSB2 (Y) cells overexpressing FBXO42 were determined by qPCR. (A-Y, n=3) Quantitative data are presented as mean±SEM from three independent experiments. P values were calculated using two-tailed Student's t-tests or analyzed using a one-way ANOVA for multiple comparisons. **P<0.01, ***P<0.001. For data in (V) and (Y), ##P<0.01 vs. FBXO42.

FIG. 11 shows RBPJ positively regulates leukemia cell invasion and tumorigenesis. (A-D) The invasion abilities of WT and RBPJ-KO JURKAT (A) and HSB2 (C) cells were measured using a 3D culture system with Matrigel. Scale bars, 50 μm. The average diameter of the spheres formed in A and C was summarized, respectively (B and D). (E-H) The anchorage-independent tumorigenesis abilities of WT and RBPJ-KO JURKAT (E) and HSB2 (G) cells were measured using a soft agar colony formation assay. The number of colonies in E and G was counted and summarized, respectively (F and H). (A-H, n=3). Quantitative data are presented as mean±SEM. P values were calculated using two-tailed Student's t-tests. **P<0.01, ***P<0.001.

FIG. 12 shows the FBXO42 ubiquitination ligase function is required for Notch signaling-dependent leukemogenesis. FBXO42-KO cells were rescued with WT FBXO42 or F-box deletion mutant and were used in the following studies. (A-C) The mRNA levels of Notch target genes in the treated HEK293T (A), JURKAT (B) and HSB2 (C) cells were determined by qPCR. (D-G) The invasion abilities of the treated JURKAT (D) and HSB2 (F) cells were evaluated using a 3D culture system with Matrigel. Scale bars, 50 μm. The respective average diameter of the spheres in D and F was summarized, respectively (E and G). (H-K) Anchorage-independent growth of the rescued JURKAT (H) and HSB2 (J) cells was measured using a soft agar colony formation assay. The number of colonies in H and J was counted and summarized, respectively (I and K). (L) JURKAT and HSB2 cells were treated with MLN4924 in a concentration-gradient manner for 36 h and then evaluated for the cell viability using cell counting kit-8 assay. (M and N) The expression of Notch target genes in MLN4924-treated JURKAT (M) and HSB2 (N) cells were determined by qPCR. (O-R) Anchorage-independent growth of MLN4924-treated JURKAT (0) and HSB2 (Q) cells was measured using soft agar colony formation assays. The number of colonies was counted and summarized (P and R). (A-R, n=3) The data are shown as the mean±SEM from the independent experiments. P values were calculated using one-way ANOVA for multiple comparisons or two-tailed Student's t-tests. *P<0.05, ** P<0.01, *** P<0.001.

FIG. 13 shows genetic and pharmacological targeting of FBXO42 attenuated leukemia progression in vivo.

(A-H) Xenograft tumor growth studies were performed with WT or FBXO42-KO JURKAT (A-D) and HSB2 (E-H) cells. Mice were euthanized 4 weeks after tumor cell injection. The tumors were excised, photographed, and weighed. The volumes (B and F) and weights (C and G) of the tumors were measured, respectively. The mRNA levels of Notch target genes in tumors were determined by qPCR, respectively (D and H). (I-K) In vivo leukemia mouse model was established by injecting WT and FBXO42-KO JURKAT cells carrying GFP into NSG mice intravenously. The percentage of GFP⁺ leukemia cells in peripheral blood was measured weekly by flow cytometry analysis (I) and summarized (K). Representative flow cytometry dot plots showing expression of GFP in peripheral blood was shown (J). (L-O) Spleens in mice from different groups were excised, a representative image is shown (L), and the spleen weight was measured (M). Tumor cell invasion was evaluated by measuring GFP intensity by fluorescence microscopy (N) and hemoxylin and eosin staining (O). Scale bars, 50 μm. (P and Q) NSG mice were transplanted with luciferase-expressing WT and FBXO42-KO JURKAT cells via tail-vein injection. Tumor growth in each group was tracked by bioluminescence imaging. (R) Survival analysis of mice from (P). (S-V) Xenograft tumor growth studies were performed with JURKAT cells. Mice bearing JURKAT xenograft were then subcutaneously administered with vehicle or 30 mg/kg MLN4924 twice daily for 21 days. At the end of study, the tumors were excised, photographed, and weighed. A macroscopic graph of the tumors is shown (R). The volumes (S) and weights (T) of the tumors and mouse weight (U) were measured. (A-O, S-V, n=5, P-R, n=10) Quantitative data are presented as mean±SEM. P values were calculated using two-tailed Student's t-tests. *P<0.05, **P<0.01, NS, not significant.

### DETAILED DESCRIPTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The articles “a”, “an”, and “the” are used herein to refer to one or to more than one (i.e., to at least one) of the grammatical object of the article. By way of example, “a polypeptide” means one polypeptide or more than one polypeptide.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Term “FBXO42” used herein refers to F-box protein 42 (Fbx42), a member of the F-box protein family. FBXO42 gene encodes a 717-amino acid protein characterized by approximately 40-aminod-acid F-box motif in its N-terminus and 3 central kelch repeats downstream of the F-box.

Term “inhibitor” used herein refers to materials capable of lowering, reducing or eliminating the amount, particular function, and particular property of a target object. Said target object can be a protein, polypeptide, nucleic acid and the like, while said inhibitor affects the amount, particular function, and particular property of the target object either directly or indirectly so as to result in the corresponding lowering, reducing or eliminating of the amount, particular function, and particular property of the target object. Said inhibitor can be a protein, polypeptide, nucleic acid, small molecule compound and the like.

For example, term “inhibitor” used herein refers to materials capable of lowering, reducing or eliminating the expression, transcription, translation of gene, and/or stability of protein produced therefrom, binding ability to protein etc., which includes but is not limited to a polypeptide antagonist against, inhibitory nucleotides specific to, antibodies against protein, small molecule compound inhibitors capable of inhibiting activity, and/or materials capable of inhibiting the interaction between protein and other membrane proteins, and the like.

For example, term “FBXO42 specifc inhibitor” used herein refers to materials capable of lowering, reducing or eliminating the expression, transcription, translation of FBXO42 gene, and/or stability of FBXO42 protein produced therefrom, binding ability to protein etc., which includes but is not limited to a polypeptide antagonist against FBXO42, inhibitory nucleotides specific to FBXO42, antibodies against FBXO42 protein, small molecule compound inhibitors capable of inhibiting FBXO42 activity, and/or materials capable of inhibiting the interaction between FBXO42 protein and ligands, and the like.

Term “antibody” used herein refers to any immunoglobulin or complete molecule and fragments thereof which binds to a specific epitope. Said antibody includes but not limited to polyclonal antibodies, monoclonal antibodies, chimeric antibodies, humanized antibodies, single chain antibodies, and fragments and/or parts of intact antibodies, as long as such fragments or parts retain the antigen binding capacity of the parent antibody. In this disclosure, for example, “antibody against FBXO42” refers to monoclonal antibodies, polyclonal antibodies, single chain antibodies and immunological activie fragments or parts thereof capable of specific binding to FBXO42 protein, or functional variants or functional fragments thereof. In this disclosure, terms such as “FBXO42 antibody”, “antibody against FBXO42”, and “anti-FBXO42 antibody” are used interchangeably.

In this disclosure, “functional variant” refers to the protein or polypeptide of the invention with one or more amino acid modification in its amino acid sequence. The modification can be a “conservative” modification (wherein the substituted amino acid has similar structure or chemical property) or a “non-conservative” modification; similar modification also include addition or deletion of amino acid or both. However, neither the modification of amino acid residue nor the addition or deletion of amino acid would substaintially change or damage the biological or immunological activity and function of the original amino acid sequence. In this disclosure, similarly, “functional fragment” refers to any part of the protein or polypeptide of the invention, which retains the substantially similar or identical biological or immunological activity and function of the protein or polypeptide of which it is a part (the parent protein or polypeptide).

Term “polynucleotide specific to FBXO42” used herein refers to nucleotide capable of binding to and/or inhibiting expression of FBXO42 gene. Typical inhibitory nucleotide includes but not limited to antisense oligonucleotides, triple helix DNAs, RNA aptamers, ribozymes, small interfering RNA (siRNA), short hairpin RNA (shRNA) and microRNA. These nucleotide compounds bind to said specific genes with higher affinity than other nucleotide sequences, so as to inhibit expression of the specific genes.

Term “small molecule compound” used herein refers to organic compounds with molecular weight less than 3 k dalton which can be either natural or chemically synthesized. Term “derivative” used herein refers to compounds generated by modifying the parent organic compound through one or more chemical reactions, which have similar structures as the parent organic compound and similar effects in their functions. Term “analogue” used herein refers to compounds which were not generated by chemically modifying the parent organic compound but are similar to the parent organic compound in structure and have similar effects in their functions.

Term “disease” used herein refers to Notch signaling dependent disease e.g. Notch signaling acitivated cancers. Notch signaling-dependent disease include activating mutations and/or amplification of Notch gene and/or Notch pathway activity. The cancer can be but not limited the T-acute lymphoblastic leukemia (Activating mutations of NOTCH1 in human T cell acute lymphoblastic leukemia. Science. 2004; 306: 269-71. CUTLL1, a novel human T-cell lymphoma cell line with t (7; 9) rearrangement, aberrant NOTCH1 activation and high sensitivity to gamma-secretase inhibitors. Leukemia. 2006; 20: 1279-87), Chronic lymphocytic leukemia (NOTCH1 mutations influence survival in chronic lymphocytic leukemia patients. BMC Cancer. 2013; 13: 274), Multiple myeloma (Inhibition of Notch signaling induces apoptosis of myeloma cells and enhances sensitivity to chemotherapy. Blood. 2008; 111: 2220-9), lymphoma e.g. Hodgkin lymphoma (Activated Notch1 signaling promotes tumor cell proliferation and survival in Hodgkin and anaplastic large cell lymphoma. Blood. 2002; 99: 3398-403.), Burkitt lymphoma (Notch is an essential upstream regulator of NF-kappaB and is relevant for survival of Hodgkin and Reed-Sternberg cells. Leukemia. 2012; 26: 806-13), Diffuse large B-cell lymphoma (Gain-of-function mutations and copy number increases of Notch2 in diffuse large B-cell lymphoma. Cancer Science. 2009; 100: 920-926.), Mantle cell lymphoma (Whole transcriptome sequencing reveals recurrent NOTCH1 mutations in mantle cell lymphoma. Blood. 2012; 119: 1963-1971), Splenic marginal zone lymphoma (The coding genome of splenic marginal zone lymphoma: activation of NOTCH2 and other pathways regulating marginal zone development. J Exp Med. 2012; 209: 1537-51.), Follicular lymphoma (Molecular detection of t (14; 18) (q32; q21) in follicular lymphoma. Methods Mol Biol. 2013; Recurrent Mutations of NOTCH Genes in Follicular Lymphoma. Blood. 2013; 122: 4253), breast cancer (Notch1 is involved in migration and invasion of human breast cancer cells), liver cancer (Differentiation-inducing therapeutic effect of Notch inhibition in reversing malignant transformation of liver normal stem cells via MET. Oncotarget 9, 18885-18895 (2018).), lung cancer (Alterations of the Notch pathway in lung cancer. Proc. Natl Acad. Sci. USA 106, 22293-22298 (2009).), lung adenocarcinoma cells (Notch-1 stimulates survival of lung adenocarcinoma cells during hypoxia by activating the JGF-1R pathway. Oncogene 29, 2488-2498 (2010). Oxygen concentration determines the biological effects of NOTCH-1 signaling in adenocarcinoma of the lung. Cancer Res. 67, 7954-7959 (2007).). Preferably, the Notch signaling-dependent disease is selected from leukemia, myeloma, lymphoma, breast cancer, liver cancer, head and neck squamous cell carcinoma (HNSCC), lung cancer and other cancers carrying the activating mutations and/or amplification of Notch gene and/or Notch pathway activity.

Term “therapeutic target” used herein refers to various materials that can be used to treat a certain disease and the target of the material in animal or human bodies. Treatment effects on said disease are obtainable when said materials act on said target. Said materials can be a variety of materials such as protein, polypeptide, nucleic acid, small molecule compound, said target can be material substances such as a certain gene (including a specific sequence of a gene), a ceratin protein (including a specific site of a protein), a certain protein complex (including specific binding site thereof), or certain charactistics, certain functions, certain interaction relationships with peripheral substances and environment of aforementioned genes and/or proteins, etc, as long as said materials can affect the gene, protein, protein complex, or charactistic, function, interaction relationship thereof so as to treat the disease.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

Terms “treat”, “treating”, or “treatment” used herein refer to reversing, ameliorating or inhibiting the progression of the disease to which the term is applied, or one or more symptoms of the disease. As used herein, depending on the condition of the patient, the term also include prevention of disease, which includes the prevention of disease or the onset of any symptoms associated therewith, and ameliorating symptoms or reducing the severity of any condition before its onset.

Terms “inhibit”, “weaken”, “down-regulate”, “remove” and the like all refer to reduction or decreasing in quantity or degree. Such reduction or decreasing is not limited to any extent as long as it exhibits such a trend. For example, the reduction or decreasing can be 100% relative to the original quantity or degree, or can be 50% or even 1% or less.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215: 403-410 (1990); Stephen F. et al., Nucleic Acids Res., 25: 3389-3402 (1997)), ClustalW2 (available on the website of European Bioinformatics Institute, see also, Higgins D. G. et al., Methods in Enzymology, 266: 383-402 (1996); Larkin M. A. et al., Bioinformatics (Oxford, England), 23 (21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

The present disclosure will be further illustrated in detail below. However, ways to carry out the present invention are not limited to the following examples.

### Experimental Model and Subject Details

### Constructs

Genes encoding RBPJ and FBXO42 were amplified from cDNAs by PCR and cloned into a pDONR201 vector (Invitrogen, Carlsbad, CA) as entry clones and subsequently transferred to Gateway-compatible destination vectors for the expression of C-terminal SFB (cSFB)- or MYC-tagged fusion proteins. Deletion mutants of FBXO42 and RBPJ were generated by introducing point mutations and were verified by sequencing.

### Cell Culture and Transfection

HEK293T cells were cultured in DMEM supplemented with 10% fetal bovine serum and 1% penicillin/streptomycin (Thermo Fisher Scientific). HSB2 and JURKAT cells were cultured in RPMI 1640 medium supplemented with 10% fetal bovine serum and 1% penicillin/streptomycin in a humidified incubator with 5% CO₂ at 37° C.

To establish HEK293T cells stably expressing cSFB-RBPJ and cSFB-FBXO42, the cells were transfected with the respective plasmids using polyethylenimine (Polysciences) and selected in DMEM supplemented with 2 μg/mL puromycin (Sangon, China) for at least 2 weeks.

For KO experiments, CRISPR constructs were packaged into lentiviruses by cotransfecting them with the packaging plasmids pMD2.G (Addgene #12259) and psPAX2 (Addgene #12260) into HEK-293T cells. Forty-eight hours after transfection, the cell medium was collected and used to infect HEK293T, HSB2 or JURKAT cells. The cells were infected twice at an interval of 24 h to achieve maximal infection efficiency.

### Generation of CRISPR-Induced KOs

A FBXO42-KO HEK293T, JURKAT and HSB2 cell lines were established by CRISPR/Cas9-mediated genome editing. The target sequences for CRISPR interference were designed using the Benchling tool (2021), ligated into a lentiCRISPR v2 plasmid (Addgene #52961) (N. E. Sanjana, O. Shalem, F. Zhang, Improved vectors and genome-wide libraries for CRISPR screening. Nat Methods 11, 783-784 (2014)) at the BsmBI restriction site and packaged into lentivirus via cotransfection with the packaging plasmids pMD2.G and psPAX2 in HEK293T cells. HEK293T, JURKAT and HSB2 cells were infected with lentiCRISPR virus at the desired titer and then selected with puromycin. Individual clones were further expanded, and the loss of target protein expression was confirmed by immunoblotting.

The sgRNA sequence for FBXO42: 5′-CGGCCCTTGTCTGCAAACAG (SEQ ID NO: 1); RBPJ: 5′-AAAGAACAAATGGAACGCGA (SEQ ID NO: 2).

Western Blotting and Immunoprecipitation Cells were washed twice with phosphate-buffered saline (PBS) and dissolved in NETN lysis buffer (20 mM Tris-HCl, pH 8.0; 100 mM NaCl; 0.5% NP-40; and 1 mM EDTA) supplemented with protease and phosphatase inhibitors (Sangon, China). Whole-cell lysates were subjected to SDS-PAGE and were then immunoblotted with specific antibodies.

For immunoprecipitation, 1×10⁷ cells were lysed with NETN buffer on ice for 30 min. The lysates were then incubated with 30 μL of conjugated S-beads (for SFB-tagged pull-down assay) for 2 h at 4° C. or incubated with antibodies against endogenous proteins for 1 h at 4° C. followed by the addition of 20 ptL of protein A/G agarose and incubation for 2 h at 4° C. The immunoprecipitates were washed with lysis buffer three times before immunoblot analysis. The following primary antibodies were used: rabbit anti-RBPJ (5313S, CST, RRID:AB_2665555), mouse anti-FBXO42 (TA800283, OriGene, RRID:AB_2625356), THE™ HA Tag (A01244, Genscript), THE™ c-MYC Tag (A00704, Genscript), ANTI-FLAG® M2 antibody (B3111, Sigma-Aldrich, RRID:AB_2910145), rabbit anti-Ubiquitin (AF0306, Beyotime), rabbit anti-β-Actin (AC026, ABclonal, RRID:AB_2768234), rabbit anti-LSD1 (YM0422, Immunoway), rabbit anti-SMARCA4 (ET1611-85, HuaBio), rabbit anti-SMARCA2 (ER65406, HuaBio), rabbit anti-SMARCC2 (ER62787, HuaBio), rabbit anti-ORC2 (A15697, ABclonal). The following secondary antibodies were used: Goat Anti-Mouse IgG Antibody (H&L) [HRP](A00160, Genscript), Goat Anti-Rabbit IgG Antibody (H&L) [HRP](A00178, Genscript).

### TAP

TAP purification was performed as described previously (W Bian et al., Low-density-lipoprotein-receptor-related protein 1 mediates Notch pathway activation. Dev Cell 56, 2902-2919 e2908 (2021)). Briefly, 1×10⁸ HEK293T cells stably expressing cSFB-RBPJ or FBXO42 were lysed in 5 ml of NETN buffer (with protease inhibitors) at 4° C. for 30 min followed by TurboNuclease treatment. The lysate was then incubated with streptavidin-conjugated beads (Thermo Fisher Scientific, Waltham, MA) for 2 h at 4° C. After washing with NETN buffer, the bound proteins were eluted with NETN buffer containing 2 mg/mL biotin (Sigma, St. Louis, MO) for 2 h at 4° C. The eluates were then incubated with S-protein beads (EMD Millipore, Burlington, VT) for 4 h. The beads were washed three times with NETN buffer and subjected to SDS-PAGE, followed by Coomassie blue staining. The whole band was excised and subjected to in-gel trypsin digestion and MS analysis.

### In Vivo Ubiquitination Assay

For the in vivo ubiquitination assay, HEK293T cells were transfected with the indicated plasmids and treated with or without 10 μM MG132 (S2619, Selleck) for 4 h before harvest. Whole cells were lysed with NETN buffer containing protease inhibitors. Equal amounts of protein lysates were pulled down with S-protein beads for 4 h at 4° C. After incubation, the beads were extensively washed three times with NETN buffer, boiled with sample buffer for min and subjected to SDS-PAGE followed by immunoblotting with antibodies against various proteins as indicated. For endogenous RBPJ ubiquitination detection, the lysate was immunoprecipitated with RBPJ antibody, and then immunoblot with antibody against ubiquitin.

### Quantitative Real-Time PCR

Total RNA was isolated from cells using TRIzol reagent (Takara), and cDNA synthesis was performed using 1 μg of total RNA with HiScript III reverse transcriptase (R212-02, Vazyme). The levels of mRNA for the specific genes were quantified by SYBR green qPCR according to the manufacturer's guidance on a Jena Qtower3G quantitative PCR system. The relative mRNA levels were determined using the comparative Ct method with Actin as the reference gene following the formula 2^{-ΔΔCt}. The primers used are listed:

HES1-F:
(SEQ ID NO: 3)
5′-CCTGTCATCCCCGTCTACAC,
HES1-R:
(SEQ ID NO: 4)
5′-CACATGGAGTCCGCCGTAA,
HES5-F:
(SEQ ID NO: 5)
5′-CGCATCAACAGCAGCATCGAG,
HES5-R:
(SEQ ID NO: 6)
5′-GACGAAGGCTTTGCTGTGCT,
c-MYC-F:
(SEQ ID NO: 7)
5′-GGCTCCTGGCAAAAGGTCA,
c-MYC-R:
(SEQ ID NO: 8)
5′-CTGCGTAGTTGTGCTGATGT,
Actin-F:
(SEQ ID NO: 9)
5′-TTGCCGACAGGATGCAGAAGGA,
Actin-R:
(SEQ ID NO: 10)
5′-AGGTGGACAGCGAGGCCAGGAT.

### Luciferase Reporter Assay

Luciferase reporter constructs containing the HES1 and HES5 promoters and 8×RBPJ-binding sites were generated by inserting the HES1 and HES5 promoters and the 8×RBPJ binding site sequence into the pGL3-basic luciferase vector upstream of the firefly luciferase gene. For the luciferase assay, HEK293T cells were plated at 50% confluency in 24-well plates and grown overnight. The firefly luciferase reporter construct and the Renilla control reporter were cotransfected into the cells at a molar ratio of 10:1. After 24 h of culture, the luciferase activity was assayed with the Dual Luciferase assay kit (11402ES60, YEASEN) with normalization to Renilla activity.

### Immunofluorescence Staining

Cells were seeded in a cell culture plate, fixed with 4% paraformaldehyde at room temperature for 10 min, permeabilized 10 min with 0.1% Triton X-100, washed with PBS and blocked in 5% BSA in PBS for 30 minutes before labelling in anti-HP1 alpha primary antibody (ab109028, Abcam, RRID:AB_10858495) at room temperature for 1 h. After incubation, cells were washed with PBS twice, stained with goat-anti-rabbit Alexa Fluor®488-labelled IgG (ab150077, Abcam, RRID:AB_2630356) at room temperature for 1 h, and subjected to 4′,6-diamidino-2-phenylindole (DAPI) staining (4083S, CST,). Coverslips were mounted using FluorSave™ Reagent (345789, Milipore). The cells were viewed using an Olympus FV3000 Microscope Imaging System (Olympus, Japan).

### Chromatin Fractionation

To isolate cytoplasm and chromatin fractions, WT and FBXO42 KO HEK293T or leukemia cells were harvested and fractionated as previously described (W Bian et al., Low-density-lipoprotein-receptor-related protein 1 mediates Notch pathway activation. Dev Cell 56, 2902-2919 e2908 (2021); T Tian et al., The ZATT-TOP2A-PICHAxis Drives Extensive Replication Fork Reversal to Promote Genome Stability. Mol Cell 81, 198-211 e196 (2021)) with slight modifications. Briefly, cells were resuspended in cold buffer A (10 mM HEPES (pH 7.9), 10 mM KCl, 1.5 mM MgCl2, 0.34 M sucrose, 10% glycerol, 1 mM dithiothreitol (DTT), 0.1% Triton X-100) containing protease inhibitors for 5 min at 4° C. Lysates were centrifuged at 1,500×g for 5 min, the supernatant was further clarified by high-speed centrifugation (13,000×g, 10 min, 4° C.) to remove cell debris and insoluble aggregates, and collected as the cytoplasm fraction. The nuclei were washed once with buffer A without 0.1% Triton X-100 and then lysed in Buffer B (3 mM EDTA, 0.2 mM EGTA, 1 mM DTT) containing protease inhibitors for 10 min at 4° C. the soluble nuclear proteins were separated from chromatin by centrifugation (2,000×g, 5 min). Isolated chromatin-enriched pellets were washed once with buffer B and spun down at high speed (13,000×g, 1 min) followed by lysed in 2×Laemmli sample buffer. The samples were then subjected to SDS-PAGE followed by immunoblotting with antibodies against various proteins as indicated.

### CUT&Tag Assay and Sequencing

CUT&Tag assay were performed as previously described (H. S. Kaya-Okur et al., CUT&Tag for efficient epigenomic profiling of small samples and single cells. Nat Commun 10, 1930 (2019)). Briefly, 100,000 WT and FBXO42 KO JURKAT cells were collected and lysed according to manufactures' guidance (YEASEN, Cat #12597). Cell lysates were incubated at room temperature with Convanavalin A-coated magnetic beads for 1h, and then with the primary antibody against RBPJ (1:50, abcam ab25949) for 2 h, with secondary antibodies for 1 h, and with pA/G-Tn5 adapter complex for 1 h. The tagmentation takes 1 h, and DNAs were extracted using phenol-chloroform-isoamyl alcohol. Libraries were prepared using Hieff NGS® Tagment Index Kit for Illumina® (96 Index) (YEASEN, Cat #12610) and pooled together for paired-end 150-bp sequencing on an Novaseq (Novogene). Raw fastq files were trimmed using Trim Galore (length 20, e=0.1) and aligned to the human genome (hg38) using Bowtie2. Reads were sorted and converted to BAM format, data track visualization occurred using IGV. Final data analysis and visualization was performed using in house R scripts.

### MNase and DNase Sensitivity Assay

MNase and DNase sensitivity assays were performed as described previously (Y. Li et al., Histone H1 acetylation at lysine 85 regulates chromatin condensation and genome stability upon DNA damage. Nucleic Acids Res 46, 7716-7730 (2018)) with some modifications. Briefly, cell pellets were lysed in buffer A (10 mM HEPES, pH 7.9; 10 mM KCl; 1.5 mM MgCl₂; 0.34 M sucrose; 10% glycerol; 1 mM DTT; and 0.1% Triton X-100) for 10 min on ice. The nuclei were pelleted and digested with 10 U/mL MNase (2910A, Takara) in digestion buffer (10 mM Tris·HCl, pH 7.5; 1 mM NaCl; 3 mM MgCl₂; and 1 mM CaCl₂)) for 3 min at 37° C. or digested with DNase (M0303S, NEB) for 5 min at 37° C. Treated nuclei were lysed, followed by RNase A and Proteinase K digestion. Genomic DNA was purified using a DNA purification kit (DC301-01, Vazyme) and separated by 1.2% agarose gel electrophoresis. DNA bands were visualized under a Gel Doc XR+ system (Bio-Rad).

### DNase I Chromatin Accessibility Analysis

Chromatin accessibility was analyzed according to the protocol (PMID: 33654939, 30911685). Chromatin was isolated in a buffer containing 10 mM Tris-HCl (pH 7.5), 5 mM MgCl2, 1 mM CaCl2), 10 mM KCl, 300 mM sucrose, and 0.1% Triton X-100 for 5 min on ice, then washed and resuspended with the same buffer without detergent. The One third chromatin was then digested with DNase I (NEB) at 3 U/100 μL for 7 min at room temperature. Another third was treated identically without DNase I (untreated control for normalization). Reactions were stopped by addition of 10 mM EDTA/2 mM EGTA and incubated at 65° C. for 10 min. DNA was lightly sonicated, treated with 50 μg/mL RNase (Sigma) for 30 min at 37° C. and 250 μg/mL Proteinase K (Sigma) 2 h at 42° C. DNA was purified and analyzed using Jena Qtower3G system. qPCR results were analyzed according to the formula 100^{Ct(DNase I)-Ct(no DNase I)} for normalization to input DNA (no DNase I treatment).

### ATAC-Seq Library Preparation and Data Analysis

ATAC-seq library processing was performed according to the manufacture's protocol (N248, novoprotein). The procedure generally included resuspending 50,000 viable cells and isolating nuclei; then, transposition was performed using Tn5 transposase, which was followed by adaptor ligation and PCR amplification. Libraries were sequenced with 150 bp paired-end on Novaseq. All paired-end reads were first subjected to adaptor trimming using cutadapt (v1.18). Then, the clipped reads were aligned to the human genome (hg38) using bowtie2 (v2.3.3.1). Peaks were called for each sample using MACS2 (v2.1.1.20160309). ATAC-seq signal was visualized in Integrative Genomics Viewer (IGV, v2.5.3), and analyzed using deeptools (v3.3.0). Global mass spectrometry-based analysis of protein ubiquitination.

Global protein ubiquitination analysis was performed according to the manufacture's guidance (5562, CST). Briefly, the cell lysis was prepared in urea buffer, followed with reduction, alkylation, and digestion with trypsin overnight. Then the peptides were used for immunoaffinity purification using Remnant Motif (K-F-GG) and mass spectrometry detection.

### Chromatin Immunoprecipitation (ChIP)

ChIP assay was performed based on the previously described protocol (PubMed: 19632176). Cells were crosslinked with 1% formaldehyde for 10 minutes and quenched by 125 mM glycine for 5 minutes at room temperature with gentle shaking. After rinse with cold PBS twice, cells were collected in PBS supplemented with protease inhibitors, centrifuged, and lysed in ice-cold lysis buffer (1% SDS, 5 mM EDTA, 50 mM Tris-HCl pH 8.1) supplemented with protease inhibitor for 10 minutes. The cell lysate was sonicated using Bioruptor Sonicator (Diagenode) to break DNA into ˜500-bp fragments for ChIP-qPCR. Soluble chromatin was diluted in dilution buffer (1% Triton X-100, 2 mM EDTA, 150 mM NaCl, 20 mM Tris-HCl pH 8.1), and 4 μg ChIP-grade antibody was added and incubated at 4° C. for 2 h with gentle shaking. 50 μl protein A/G beads flurry (16-663, Millipore) was added and incubated for one hour at 4° C. The beads were then washed in following buffers for 10 minutes each at 4° C.: Buffer I (0.1% SDS, 1% Triton X-100, 2 mM EDTA, 150 mM NaCl, 20 mM Tris-HCl pH 8.1), Buffer II (0.1% SDS, 1% Triton X-100, 2 mM EDTA, 500 mM NaCl, 20 mM Tris-HCl pH 8.1), Buffer III (0.25 mM LiCl, 1% NP-40, 1% deoxycholate, 1 mM EDTA, 10 mM Tris-HCl pH 8.1), and TE buffer (2 times). To elude DNA, beads were incubated in elution buffer (1% SDS, 0.1 M NaHCO₃) at room temperature with aggressive shaking for 15 minutes. The supernatant was then collected and incubated at 65° C. for overnight to reverse-crosslink the DNA. DNA purification kit (DC301-01, Vazyme) was used for purifying the DNA for the subsequent qPCR. The following antibodies were used in ChIP: anti-H3K4me3 (ab8580, Abcam, RRID:AB_306649), anti-H3K27ac (ab177178, Abcam, RRID:AB_2828007), and anti-IgG (39005, CST, RRID:AB_1550038). ChIP-qPCR experiments were done in triplicates and the results were normalized to the input DNA.

### Mouse Model

All animal experiments were performed in accordance with a protocol approved by the Institutional Animal Care and Use Committee of Westlake University (AP #20-023-LX). WT and FBXO42-KO HSB2 and JURKAT cells (5×10⁶) were resuspended separately in 100 μL of Matrigel (356237, Corning) diluted with PBS at a 1:1 ratio and injected subcutaneously into the left and right flanks, respectively, of anesthetized 6- to 8-week-old female BALB/c nude mice (SLAC). Starting on day 7, tumor formation was observed biweekly. The mice were euthanized after 4 weeks of injection, and the tumors were excised, photographed, and weighed.

For the invasion assay, a leukemia model was established with NSG mice (Charles River). WT and FBXO42-KO JURKAT-GFP reporter cells (5×10⁶) were resuspended in 100 μL of PBS and injected intravenously into 6- to 8-week-old female NSG mice via the tail vein. Starting on day 7, peripheral blood leukemia cells were analyzed by detecting GFP levels with flow cytometry. At the end of the study, the mice were euthanized, and the spleen tissues were excised, photographed, fixed in 4% paraformaldehyde, paraffin-embedded and stained with hematoxylin and eosin.

For the evaluation of MLN4924 efficacy in vivo, 6- to 8-week-old female BALB/c nude mice were inoculated with 5×10⁶ JURKAT cells subcutaneously in the right flank, and tumor growth was monitored with caliper measurements. When the tumor was visible, the mice were dosed subcutaneously with vehicle or MLN4924 (30 mg/kg, twice daily) for 21 days, and tumor growth was then recorded.

### In Vivo Bioluminescence Imaging

To monitor tumor growth in living animals, JURKAT cells used for the animal studies were transduced with firefly luciferase through lentiviral infection. Then, the cells were infected with lentiCRISPR virus to knock out FBXO42, and these infected cells were engrafted intravenously into 6- to 8-week-old female NSG mice. For the imaging analysis, the animals were intraperitoneally administered 150 mg/kg D-luciferin (40902ES01, YEASEN) and anesthetized with isoflurane. Tumor luciferase images were captured with an IVIS imaging system (Biospace Imager Optima).

### Histological Analysis

Spleen tissues collected from different groups of mice were fixed in 4% paraformaldehyde and immersed in fixative for 24 h. After embedding into paraffin, 4-μm sections were prepared and placed on poly-L-lysine-coated slides. Morphological changes were analyzed by hematoxylin and eosin staining.

### Flow Cytometry Analysis

Peripheral blood was collected from NSG mice, and red blood cells were removed by RBC lysis (C3702, Beyotime). After washing the cells three times with PBS, GFP intensity was analyzed with a CytoFLEX6 flow cytometer and CytExpert software according to the manufacturer's instructions.

### Statistical Analysis

All western blotting, immunofluorescence and RT-qPCR data were obtained from at least three repeated experiments. The data were analyzed using Prism 7.0 software (GraphPad, USA) and are presented as the mean values (standard error of the mean, ±SEM). Statistical significance between two groups was determined by unpaired two-tailed Student's t test. Multiple-group comparisons were performed by one-way analysis of variance (ANOVA). P values of <0.05 (indicated with an asterisk (*) were considered significant.

### Example 1: Proteomic Analysis of the RBPJ Interaction Network Identifies FBXO42 as a Novel Regulator of Notch Signaling

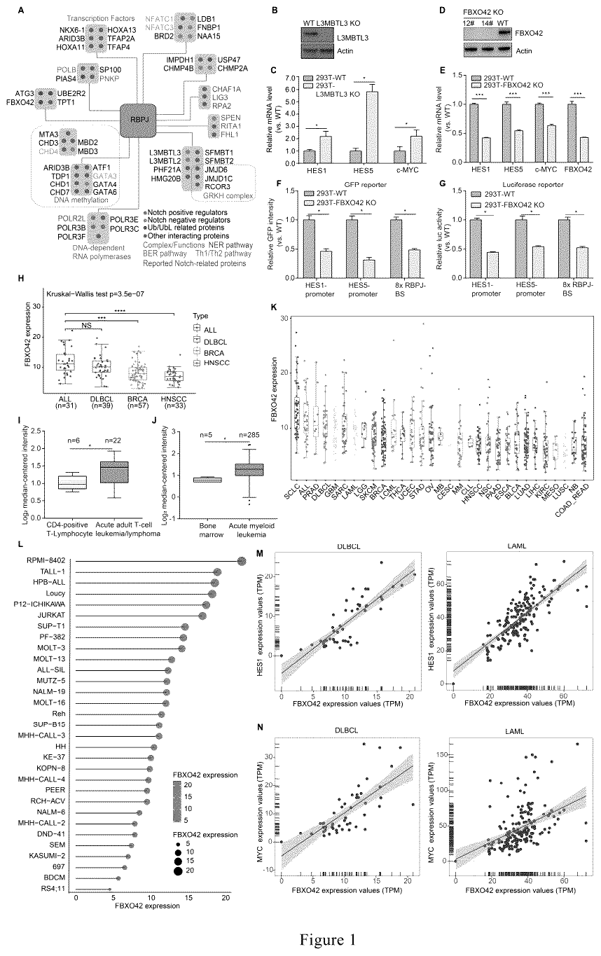

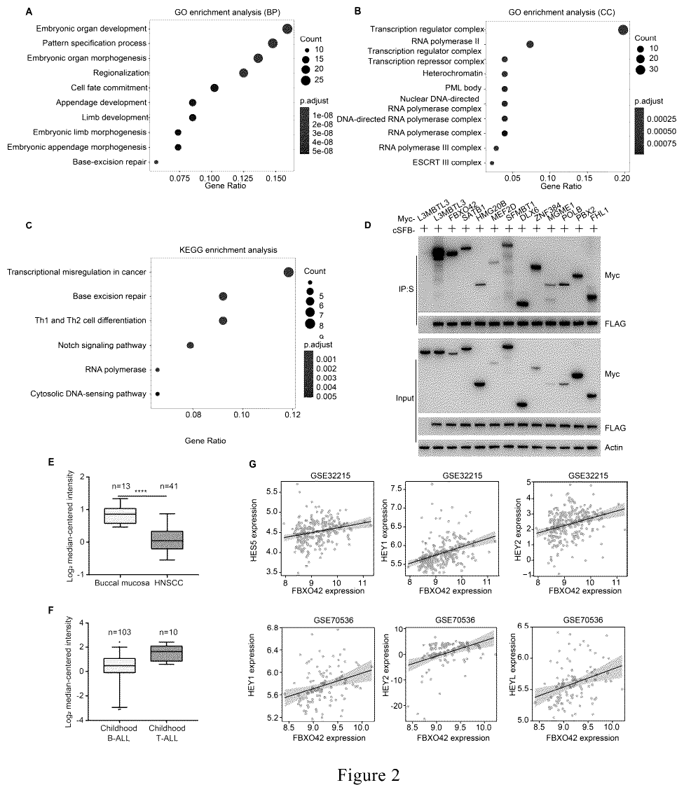

To gain a comprehensive understanding of the transcriptional regulation of the Notch pathway and identify novel RBPJ interactors, we established a RBPJ protein interaction network using TAP-MS in HEK293T cells due to its broad protein abundance and easy for transfection and manipulation. The MS analysis of purified protein extracts revealed the successful purification of RBPJ with a 570 and 381 peptide-spectrum match (PSM) against RBPJ, respectively. We analyzed the MS results using the MUSE algorithm (W Bian et al., Low-density-lipoprotein-receptor-related protein 1 mediates Notch pathway activation. Dev Cell 56, 2902-2919 e2908 (2021)) and established a high-confidence map of RBPJ interactors (FIG. 1A). Functional annotation and pathway enrichment assays showed that RBPJ interactors are highly involved in embryonic development, cell fate decisions and transcriptional regulation (FIG. 2A-C), which is consistent with their roles played in Notch signaling. We picked several of the strongest RBPJ-interacting proteins identified in this study for a coimmunoprecipitation (co-IP) assay to validate their interactions with RBPJ (FIG. 2D). All the interactors tested interacted with RBPJ, indicating that this interaction network was reliable (FIG. 2D). CRISPR/Cas9-mediated KO screening of the strongest RBPJ interactors revealed the top positive and negative regulators of Notch signaling. Knocking out L3MBTL3, a previously reported negative regulator of RBPJ (T Xu et al., RBPJ CBF1 interacts with L3MBTL3 MBT1 to promote repression of Notch signaling via histone demethylase KDM1A/LSD1. EMBO J 36, 3232-3249 (2017)), increased Notch target gene expression (FIGS. 1B and 1C), and knocking out FBXO42 significantly decreased Notch target gene expression (FIGS. 1D and 1E). Moreover, knocking out FBXO42 impaired RBPJ binding to HES1/5 promoter regions as well as constructed 8×RBPJ binding site (FIGS. 1F and 1G), indicating that FBXO42 may regulate RBPJ transcriptional activities by direct binding.

Dysregulation of Notch signaling has been linked with various cancer types, including T-ALL, DLBCL, H4NSCC and breast cancers (A. P Weng et al., Activating mutations of NOTCH1 in human T cell acute lymphoblastic leukemia. Science 306, 269-271 (2004); K. Karube et al., Integrating genomic alterations in diffuse large B-cell lymphoma identifies new relevant pathways and potential therapeutic targets. Leukemia 32, 675-684 (2018); N. Agrawal et al., Exome sequencing of head and neck squamous cell carcinoma reveals inactivating mutations in NOTCH1. Science 333, 1154-1157 (2011); N. Stransky et al., The mutational landscape of head and neck squamous cell carcinoma. Science 333, 1157-1160 (2011); D. R. Robinson et al., Functionally recurrent rearrangements of the MAST kinase and Notch gene families in breast cancer NatMed 17,1646-1651 (2011); S. Stylianou, R. B. Clarke, K. Brennan, Aberrant activation of notch signaling in human breast cancer Cancer Res 66, 1517-1525 (2006)). We found that FBXO42 was highly expressed in Notch-activated T-cell leukemia, DLBCL and breast cancer, and its expression was downregulated in Notch-inactivated HNSCC (FIGS. 1H-J, 2E and 1F). It was also highly expressed in various leukemia and breast cancer cell lines (FIG. 1K), especially ALL cell lines (FIG. 1L). Furthermore, the expression of FBXO42 and Notch pathway target geneHES1, MYC, HES5, HEY1, HEY2, HEYL showed a relatively high correlation in DLBCL (FIG. 1M), LAML (FIG. 1N) and ALL patients (FIG. 2G). Taken together, these data suggested a potential role of FBXO42 as an important positive regulator of Notch signaling.

### Example 2: FBXO42 Directly Interacts with RBPJ

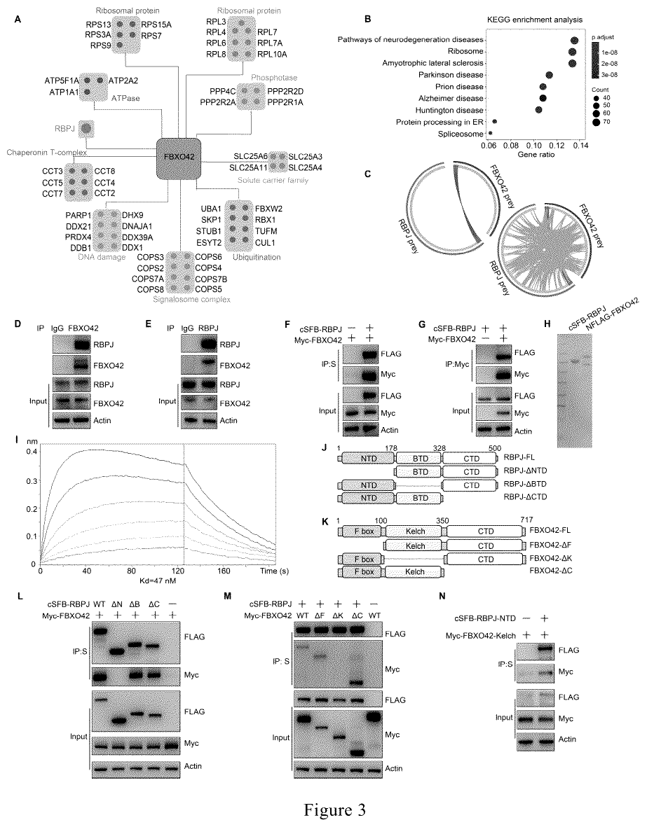

FBXO42 is a substrate-recognition component of the SKP1-CUL1-F-box protein (SCF)-type E3 ligase complex, which has been previously reported to promote p53 ubiquitination and degradation (L. Sun et al., JFK, a Kelch domain-containing F-box protein, links the SCF complex to p53 regulation. Proc Natl Acad Sci USA 106,10195-10200 (2009)). To determine whether RBPJ and FBXO42 directly interact, we performed reciprocal TAP-MS using FBXO42 as the bait and established an FBXO42 interaction network (FIG. 3A). A Kyoto Encyclopedia of Genes and Genomes (KEGG) enrichment analysis indicated the potential involvement of FBXO42 in many pathological conditions (FIG. 3B). We identified several previously reported FBXO42 interactors, including SKP1, CUL1, and COPS family members, which are involved in the deneddylation of the cullin subunits in SCF-type E3 ligase complexes (S. Lyapina et al., Promotion ofNEDD-CUL1 conjugate cleavage by COP9 signalosome. Science 292, 1382-1385 (2001); R. Groisman et al., The ubiquitin ligase activity in the DDB2 and CSA complexes is differentially regulated by the COP9 signalosome in response to DNA damage. Cell 113, 357-367 (2003)). RBPJ has also been repeatedly identified as a strong interactor of FBXO42 (FIG. 3A), indicating that FBXO42 forms a stable protein complex with RBPJ. Interestingly, although there is little overlap between RBPJ- and FBXO42-interacting proteins, the functions of these proteins overlap to a high degree (FIG. 3C), indicating that FBXO42 may specifically facilitate RBPJ transcriptional activity.

We further validated the interaction between FBXO42 and RBPJ using antibodies against endogenous FBXO42 or RBPJ (FIGS. 3D and 3E), as well as epitope-tagged RBPJ and FBXO42 (FIGS. 3F and 3G). FBXO42 and RBPJ strongly interacted with each other (FIGS. 3D-G). To estimate the dynamic binding parameters that underlie the RBPJ/FBXO42 interaction in vitro, we performed a biomolecular interaction analysis with purified recombinant RBPJ and FBXO42 proteins (FIG. 3H). FBXO42 interacted with RBPJ with very high affinity (Kd=47 nM) in vitro (FIG. 3I). To identify the binding regions on RBPJ and FBXO42, we generated a series of domain deletion mutants of RBPJ and FBXO42 (FIGS. 3J and 3K). We found that the N-terminal domain (NTD; amino acids [aa]1-178) of RBPJ (FIG. 3L) and the Kelch domain (aa 101-350) of FBXO42 (FIG. 3M) are critical for their interaction. Consistently, a strong interaction between RBPJ-NTD and FBXO42-Kelch domain was observed (FIG. 3N). Taken together, these data demonstrated the direct interaction between RBPJ and FBXO42 both in vitro and in cells, which was mediated by the NTD of RBPJ and the Kelch domain of FBXO42.

### Example 3: FBXO42 Promotes RBPJ K63-Linked Polyubiquitination and Positively Regulates Notch Signaling

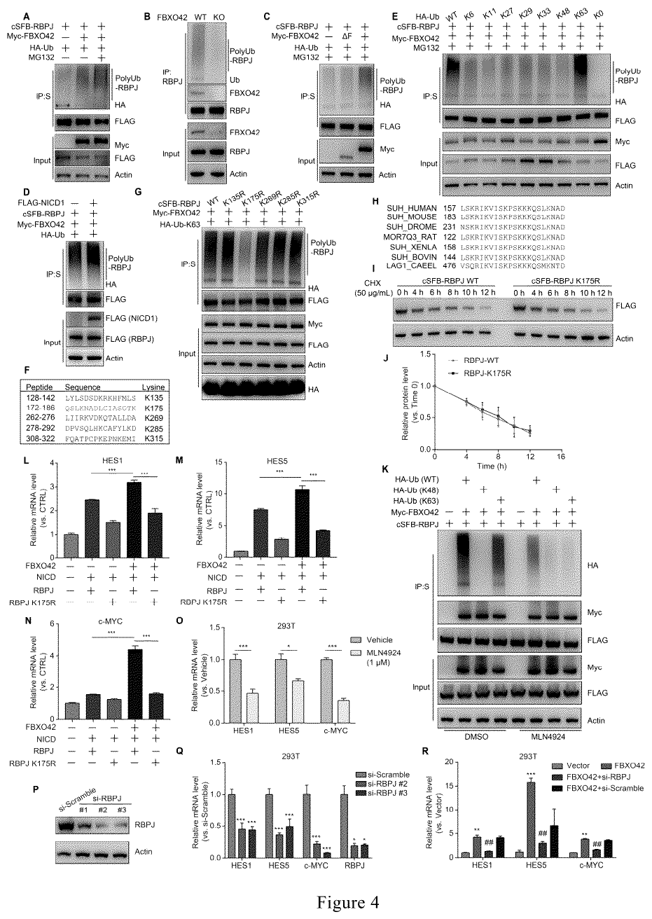

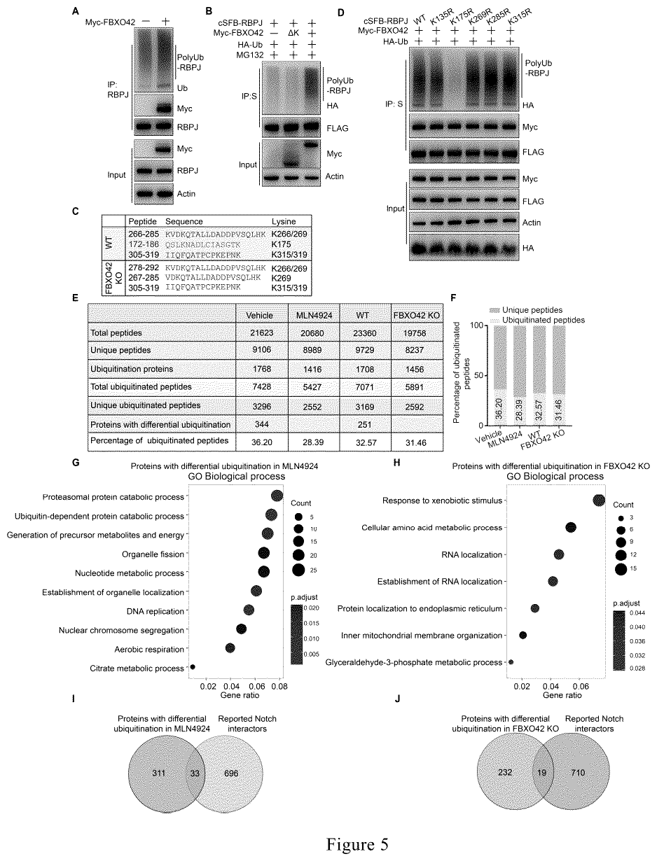

As FBXO42 belong to the SCF complex, we wondered whether FBXO42 is involved in the ubiquitination of RBPJ. Indeed, we found that FBXO42 promoted RBPJ polyubiquitination, which was markedly attenuated after FBXO42 depletion (FIGS. 4A and 4B, 5A). The FBXO42 F-box domain, which links FBXO42 to other components in the SCF complex, was required for RBPJ polyubiquitination (FIG. 4C). The Kelch domain of FBXO42, which mediates its interaction with RBPJ, was also required for RBPJ polyubiquitination (FIG. 5B). Overexpressing NICD slightly increased RBPJ polyubiquitination, suggesting a potential role of Notch signaling activation or upregulation in promoting RBPJ polyubiquitination (FIG. 4D). Using ubiquitin mutants in which only a single wild-type (WT) K residue was retained while all the other K residues were replaced with arginine (R) residues, we found only overexpressing K63 ubiquitin with FBXO42 promoted substantial RBPJ polyubiquitylation, indicating that FBXO42 specifically promotes RBPJ K63-linked polyubiquitination (FIG. 4E).

To map the ubiquitination site(s) in RBPJ, we performed MS and analyzed the RBPJ ubiquitination profile in the presence and absence of FBXO42. Overexpression of FBXO42 greatly promoted RBPJ K175 ubiquitination, as indicated by MS (FIG. 4F). Moreover, this ubiquitin peptide was not acquired in FBXO42 KO cells (FIG. 5C). We also constructed mutants carrying K-to-R mutations in potential ubiquitination sites in RBPJ as indicated by the Phosphosite public database (https://www.phosphosite.org) and detected their ubiquitination intensity. Only the K175R mutant significantly abrogated FBXO42-mediated RBPJ polyubiquitination (FIGS. 4G and 5D). The K175 residue is evolutionally conserved, suggesting that homologous sites in other organisms may be similarly modified (FIG. 4H). K175 ubiquitination did not affect the turnover rate of RBPJ, indicating that it does not mediate RBPJ proteolytic degradation (FIGS. 41 and 4J). Since FBXO42 is the substrate-recognizing component in the SCF complex, we utilized MLN4924, a small-molecule inhibitor of the NEDD8-activating enzyme, to determine whether the function of FBXO42 can be pharmacologically targeted. MLN4924 inhibits Cullin-1 neddylation and thus SCF activity and is currently in phase I-III clinical trials (T A. Soucy et al., An inhibitor of NEDD8-activating enzyme as a new approach to treat cancer Nature 458, 732-736 (2009); T A. Soucy, P G. Smith, M. Rolfe, Targeting NEDD8-activated cullin-RING ligases for the treatment of cancer Clin Cancer Res 15, 3912-3916 (2009)). Indeed, MLN4924 effectively abrogated the FBXO42-mediated K63-linked polyubiquitination of RBPJ (FIG. 4K). Besides, to detect whether there are other Notch pathway proteins affected by FBXO42. we analyzed the global ubiquitination changes upon FBXO42 knockout and MLN4924 treatment and found that percentage of ubiquitinated peptides were decreased in FBXO42 knockout and MLN4924 group (FIGS. 5E and 5F). Proteins with differential ubiquitination upon MLN4924 treatment mainly involved in proteasome and ubiquitin process (FIG. 5G), while in FBXO42 knockout cell, the differential proteins are involved in RNA process (FIG. 5H). Interestingly, 33 and 19 of these proteins were also found in reported Notch interactors (FIGS. 5I and 5J), suggesting a relevance of FBXO42 in Notch signaling.

Ubiquitin conjugation via the K48 linkage is a mark that targets modified proteins for proteasomal degradation, whereas K63-linked conjugation often plays a role in signal transduction (C. M. Pickart, D. Fushman, Polyubiquitin chains: polymeric protein signals. Curr Opin Chem Biol 8, 610-616 (2004)). Considering that RBPJ is the main transcription factor in Notch signaling, FBXO42 may regulate Notch pathway activation by promoting RBPJ K63-linked ubiquitination. Indeed, overexpressing FBXO42 with WT RBPJ, but not the RBPJ K175R mutant, significantly increased the expression of the Notch target genes HES1, HES5 and c-MYC (FIG. 4L-N), indicating that RBPJ K175 polyubiquitination is required for its transcriptional activity. MLN4924 treatment also suppressed the expression of the aforementioned Notch target genes (FIG. 40), supporting that FBXO42-mediated K63-linked polyubiquitination of RBPJ is involved in Notch signaling activation or upregulation. Knocking down RBPJ expression decreased the expression of the Notch target genes HES1, HES5, and c-MYC (FIGS. 4P and 4Q) and abolished the FBXO42-promoted activation of these genes (FIG. 4R), indicating that FBXO42-promoted Notch activation is RBPJ-dependent.

Together, these findings suggested that FBXO42 positively regulated Notch signaling by promoting K63-linked polyubiquitination of RBPJ at K175.

### Example 4: FBXO42 Regulates RBPJ Chromatin Association and Transcriptional Activity

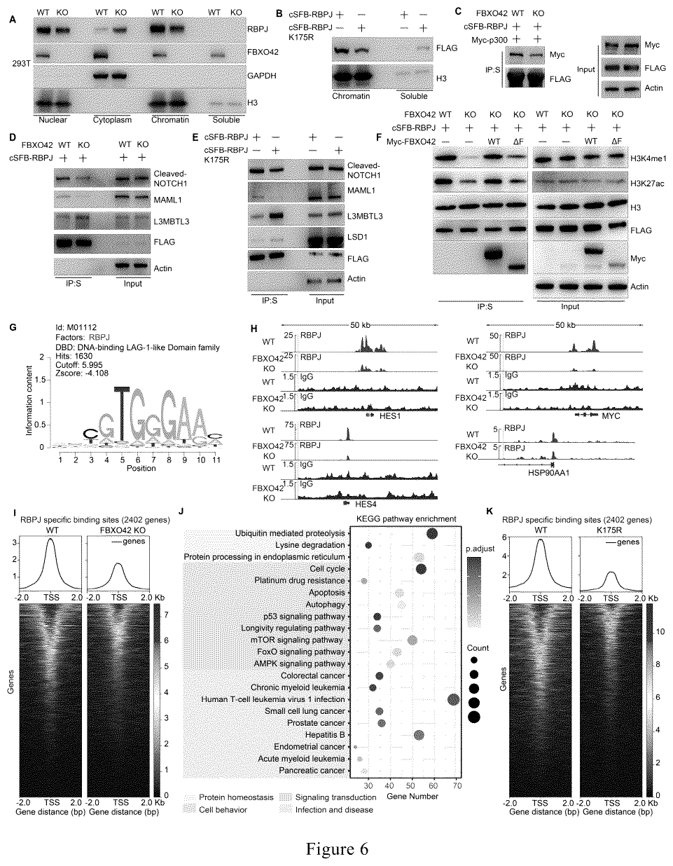

RBPJ is considered to play a dual role in the regulation of Notch signaling. Depletion of RBPJ can result in either the inhibition or activation of Notch target genes, depending on the cellular context (R. Kopan, M. X. Ilagan, The canonical Notch signaling pathway: unfolding the activation mechanism. Cell 137, 216-233 (2009)). To further illustrate the mechanism of RBPJ transcriptional activity regulation, we performed a subcellular fractionation assay and evaluated the level of RBPJ in different cellular compartments. Knocking out FBXO42 decreased the levels of nuclear and chromatin-bound RBPJ while increasing the cytoplasmic RBPJ level (FIG. 6A). The RBPJ K175R mutant also showed less chromatin binding than WT RBPJ (FIG. 6B), suggesting that FBXO42-mediated polyubiquitination of RBPJ regulated RBPJ association with chromatin.

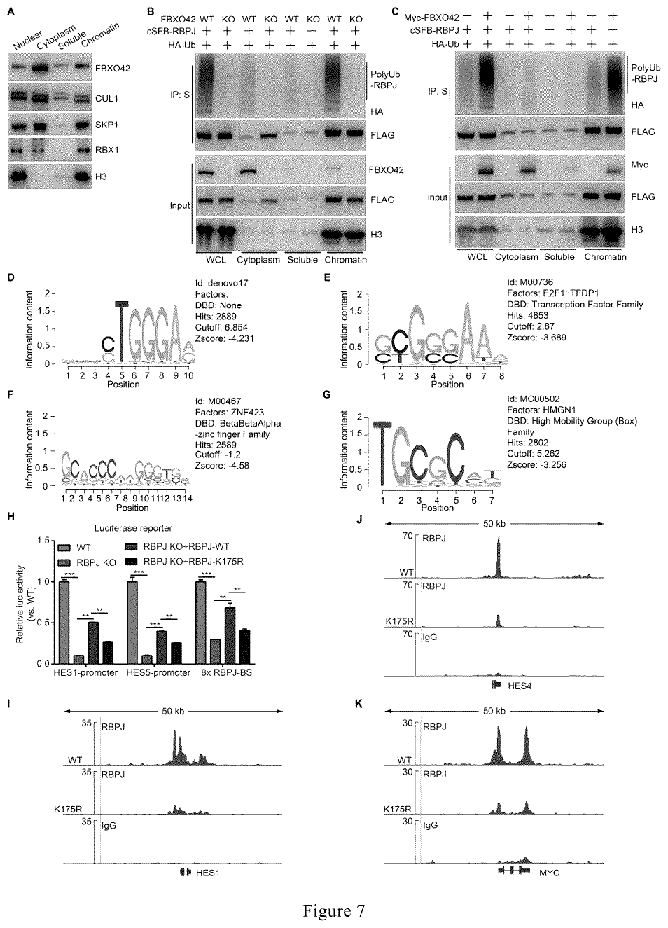

To further explore the molecular mechanism by which RBPJ transcription is activated, we evaluated the role of FBXO42 in RBPJ cofactor selectivity because the transcriptional activity of RBPJ depends on its interaction with coactivators or corepressors (K. Tanigaki, T Honjo, Two opposing roles of RBP-J in Notch signaling. Curr Top Dev Biol 92, 231-252 (2010)). Knocking out FBXO42 suppressed the interaction of RBPJ with the coactivators p300, MAML1, and NICD1 while enhancing its interaction with the corepressor L3MBTL3 (FIGS. 6C and 6D). The RBPJ K175R mutant showed a cofactor selectivity similar to that after FBXO42 KO (FIG. 6E), indicating that FBXO42-mediated polyubiquitination of RBPJ regulates RBPJ cofactor preference. Next, we wondered whether FBXO42 directly modulates RBPJ transcriptional activity. Knocking out FBXO42 expression suppressed the histone 3 (H3) K4 methylation and H3K27 acetylation levels of RBPJ, which were rescued by overexpressing WT FBXO42 but not by overexpressing the FBXO42 mutant with it's the F-box deleted (FIG. 6F). To evaluate the transcription activity, we performed CUT&Tag assay of RBPJ in WT and FBXO42 KO cells. In addition to the classical RBPJ motif, other leukemia relevant motifs were also identified (FIGS. 6G and 7D-G). Especially, we found that knocking out FBXO42 led to a decrease in global RBPJ binding and the chromatin recruitment of RBPJ to its target genes HES1, HES4, and MYC (FIGS. 6H and 6I). Genes with differential RBPJ binding affinity after FBXO42 KO were mainly enriched in protein homeostasis, cell behavior, signaling transduction and Notch-related cancers, consistent with the biological role played by RBPJ (FIG. 6J). Additionally, the transcription activity of RBPJ K175R mutant was impaired as indicated by the luciferase reporter assay and CUT&Tag assay as compared with RBPJ WT (FIG. 7H-K).

Taken together, these data indicated that FBXO42-mediated polyubiquitination of RBPJ regulates RBPJ chromatin association and subsequently regulates its transcriptional activity.

### Example 5: FBXO42 Mediates Global Chromatin Remodeling in an RBPJ-Dependent Manner

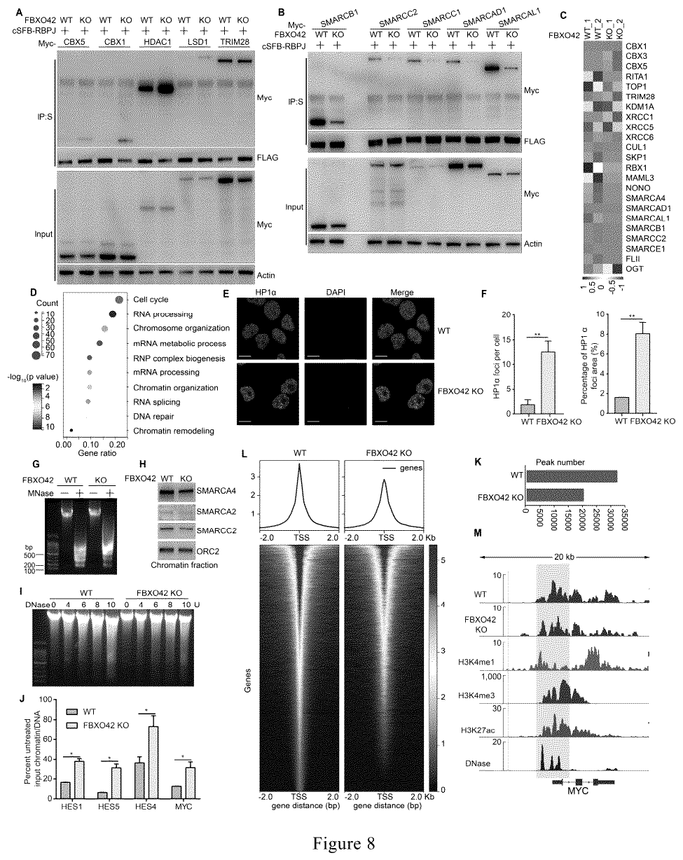

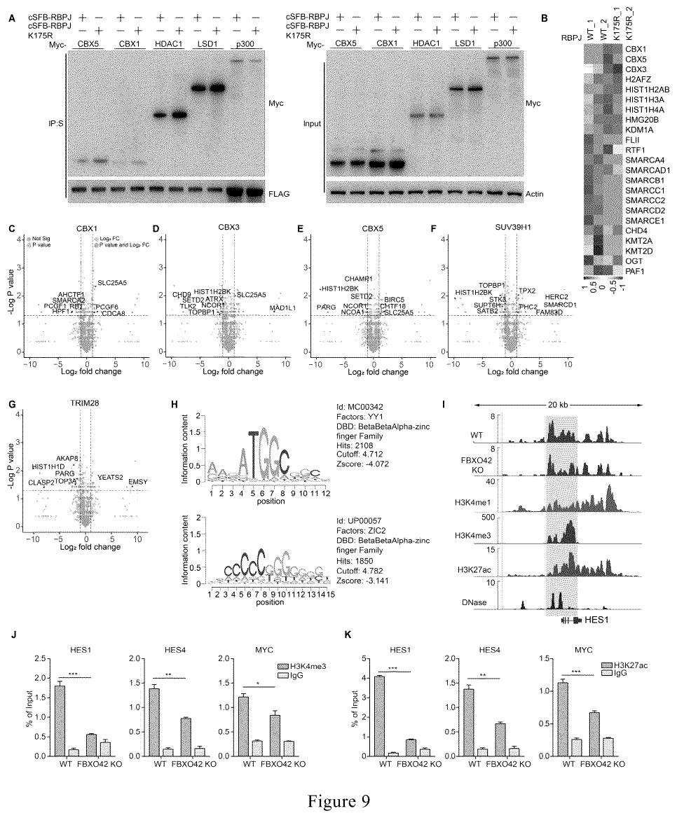

Chromatin remodeling is critical for transcriptional regulation (B. Zhang, K. J. Chambers, D. V Falle, S. Wang, Reprogramming of the SWI SNF complex for co-activation or co-repression in prohibitin-mediated estrogen receptor regulation. Oncogene 26, 7153-7157 (2007); B. B. Liau et al., Adaptive Chromatin Remodeling Drives Glioblastoma Stem Cell Plasticity and Drug Tolerance. Cell Stem Cell 20, 233-246 e237 (2017)); therefore, we wondered whether FBXO42 regulates the interactions between RBPJ and chromatin remodeling complexes. Knocking out FBXO42 broadly led to increased interactions between RBPJ and the heterochromatin components HDAC1, LSD1, TRIM28, CBX1 and CBX5, which are related to gene silencing (R. C. Allshire, H. D. Madhani, Ten principles of heterochromatin formation and function. Nat Rev Mol Cell Biol 19, 229-244 (2018)) (FIG. 8A), and decreased interactions between RBPJ and core components of the SWI/SNF complex, the chromatin remodeling complex involved in transcriptional activation (FIGS. 8B and 8C). Consistently, the RBPJ K175R mutant showed a similar interaction pattern with that of FBXO42 KO context (FIGS. 9A and 9B), indicating that FBXO42-mediated RBPJ K175 ubiquitination was critical for its association with chromatin remodeling complexes.

To determine the overall impact of the FBXO42-RBPJ axis on chromatin remodeling activities, we analyzed the differential interactomes of the key heterochromatin components CBX1, CBX3, CBX5, SUV39Hland TRIM28 between WT and FBXO42-KO cells. Knocking out FBXO42 led to a change in the interaction landscape consisting of these heterochromatin proteins; that is, their interactions with other chromatin remodeling factors, such as EMSY, PCGF6, and PHC2, were changed (FIGS. 8D and 9C-G), further supporting their potential role in chromatin remodeling regulation.

Since these chromatin remodeling complexes are involved in chromatin compaction and relaxation, we wondered whether the FBXO42-RBPJ axis directly modulates chromatin accessibility. Indeed, the number of HP1α foci, which were heterochromatin markers, was significantly increased in FBXO42-KO cells (FIGS. 8E and 8F). Moreover, depletion of FBXO42 decreased the level of nucleosome release from chromatin after micrococcal nuclease (MNase) treatment (FIG. 8G) and the chromatin association of SWI/SNF complexes, as exemplified by an analysis of its essential ATPase subunits SWI/SNF-related matrix-associated actin-dependent regulator of chromatin A2 (SMARCA2), SMARCA4 and catalytic core subunit SMARCC2 (FIG. 8H). DNase I chromatin accessibility analysis indicated less sensitive to DNase I digestion on RBPJ-binding region upon FBXO42 depletion, that is more condensed in its chromatin state (FIGS. 81 and 8J). Furthermore, ATAC-seq data showed a global chromatin accessibility change (FIGS. 8K and 8L) and an effect on leukemia related transcription factors binding (FIG. 9H) after FBXO42 knockout, which was mostly related to leukemia promoter and enhancer region as characterized by H3K4mel, H3K4me3, H3K27ac ChIP-seq and DNase-seq data from ENCODE database (FIG. 8M and FIG. 9I), which was further confirmed using ChIP-qPCR (FIGS. 9J and 9K).

Taken together, FBXO42 increased global chromatin accessibly in an RBPJ-dependent manner, which may act as a modulator of RBPJ's pioneer function for Notch signaling activation or upregulation.

### Example 6: FBXO42 Plays an Essential Role in Notch Signaling-Dependent Leukemogenesis

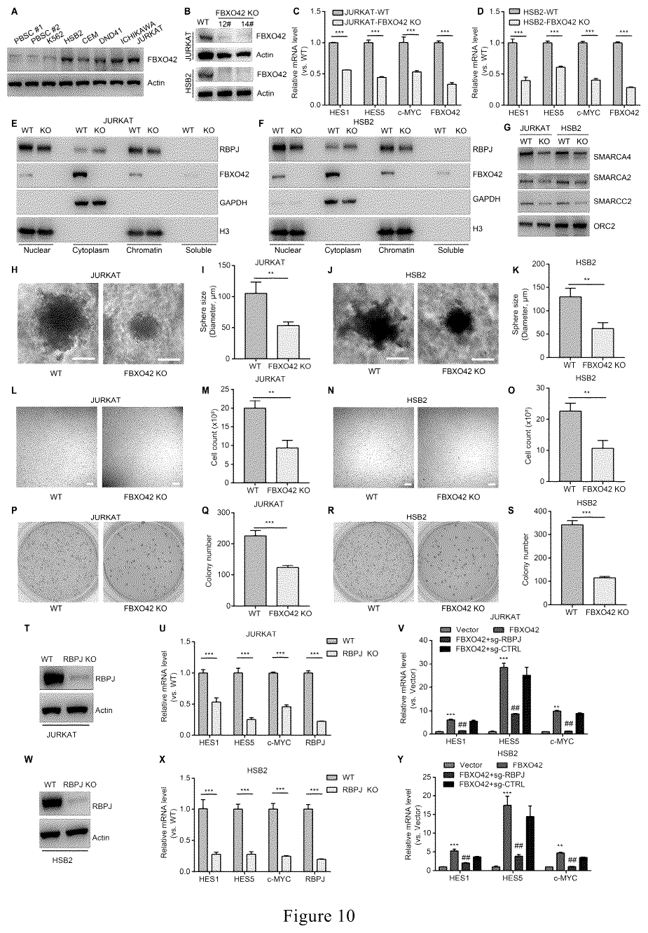

Aberrant activation of the Notch pathway is closely related to the occurrence and progression of T-ALL; however, only a subset of these patients carry NOTCH gene mutations (A. P Weng et al., Activating mutations of NOTCH1 in human T cell acute lymphoblastic leukemia. Science 306, 269-271 (2004); M. Sanchez-Martin, A. Ferrando, The NOTCH1-MYC highway toward T-cell acute lymphoblastic leukemia. Blood 129, 1124-1133 (2017)). Since FBXO42 plays a key role in Notch signaling, we wondered whether FBXO42 contributes to leukemogenesis. Therefore, we tested the protein expression in several T-ALL cell lines and selected the HSB2 and JURKAT cell lines, expressing WT NOTCH and relatively high FBXO42 levels, for subsequent studies (FIG. 10A). Knocking out FBXO42 in these two cell lines (FIG. 10B) led to decreased expression of Notch target genes (FIGS. 10C and 10D). Consistently, FBXO42 KO leukemia cells showed decreased RBPJ levels in chromatin fraction (FIGS. 10E and 10F) and reduced levels of chromatin-associated SWI/SNF complex components (FIG. 10G). To further explore the role played by FBXO42 in leukemogenesis, we evaluated the impact of FBXO42 knockout on leukemia cell invasion (FIGS. 10H-K), migration (FIGS. 10L-O), and anchorage-independent cell growth (FIGS. 10P-S). Depletion of FBXO42 significantly reduced leukemia cell invasion, migration, and tumorigenesis (FIGS. 10H-S).

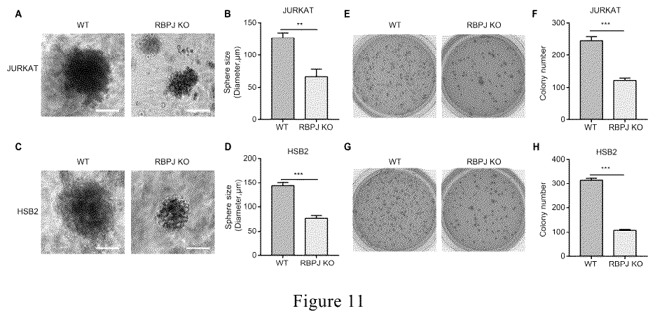

To further investigate the extent to which FBXO42 regulation of Notch signaling and leukemogenesis directly depends on RBPJ, we first analyzed the expression of Notch target genes in leukemia cells in the absence of RBPJ (FIGS. 10T and 10W). Similar to the effect of FBXO42 depletion, loss of RBPJ in the JURKAT and HSB2 cells decreased the expression of Notch target genes (FIGS. 10U and 10X). It also repressed sphere formation (FIGS. 11A-D) and anchorage-independent growth (FIGS. 11E-H), consistent with the FBXO42-KO phenotypes. We further explored the function of FBXO42 in modulating Notch signaling activity in RBPJ-deficient cells. We found that in RBPJ competent cells, the overexpression of FBXO42 led to profound upregulation of HES1, HES5 and c-MYC expression. However, in RBPJ-deficient cells, the overexpression of FBXO42 induced a mild effect on the expression of HES1, HES5 and c-MYC (FIGS. 10V and 10Y).

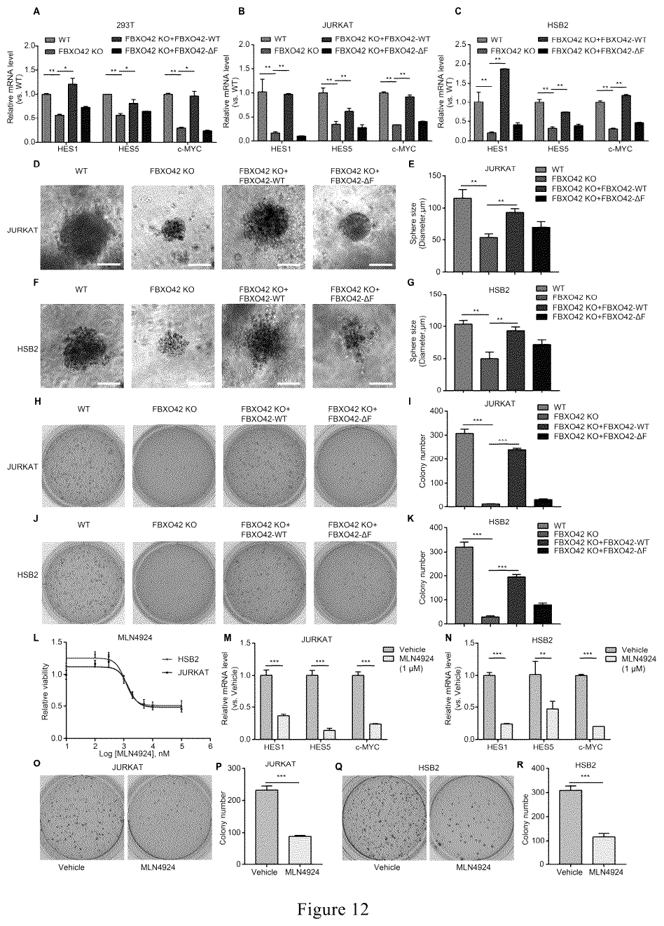

To determine whether FBXO42 regulation of leukemogenesis is dependent on its ubiquitination activity on RBPJ, we overexpressed WT FBXO42 and the FBXO42 mutant with the F-box deleted in FBXO42-KO cells and determined the rescue effect on cellular phenotypes. Overexpression of WT FBXO42 but not the F-box deletion mutant rescued Notch target gene expression in both FBXO42-deficient HEK293T and in leukemia cells (FIG. 12A-C). Moreover, the sphere formation rate (FIG. 12D-G) and anchorage-independent cell growth (FIG. 12H-K) of the leukemia cells were increased when WT FBXO42 but not the F-box deleted mutant was overexpressed. MLN4924, which abrogated FBXO42-mediated K63-linked polyubiquitination of RBPJ and Notch activation, diminished cell viability (FIG. 12L), Notch target gene expression (FIGS. 12M and 12N), and anchorage-independent growth (FIG. 12O-R) of leukemia cells, suggesting that ubiquitination activity was required for FBXO42 regulation of Notch signaling-dependent leukemogenesis.

Taken together, these data demonstrated that FBXO42 played an essential role in Notch signaling and leukemia cell tumorigenesis in a ubiquitination- and RBPJ-dependent manner.

### Example 7: Knocking Out FBXO42 Inhibits the Tumorigenesis of Human Leukemia Cells, Mouse Xenografts and Leukemia Models

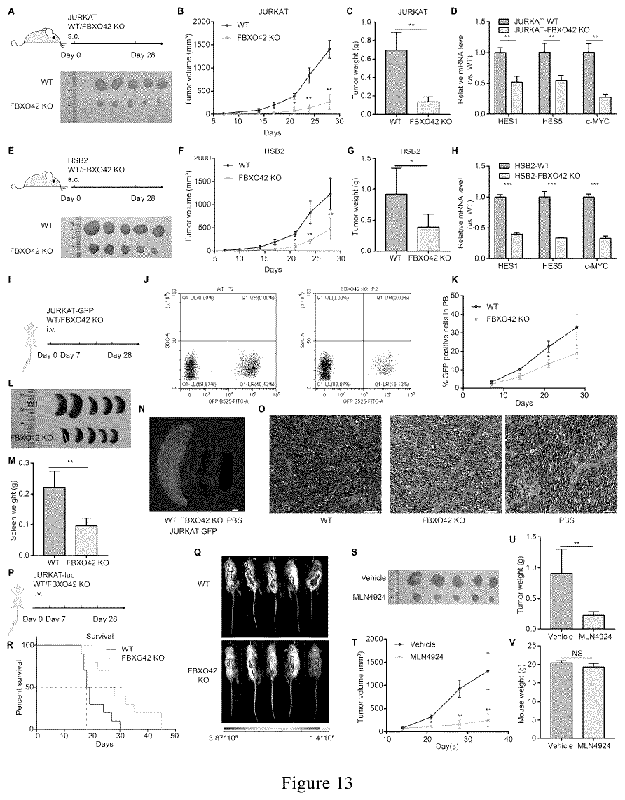

To further demonstrate the function of FBXO42 in leukemia pathogenesis, we determined the effect of FBXO42 KO on tumor formation. First, WT and FBXO42-KO JURKAT or HSB2 cells were subcutaneously injected into the left and right flanks of 6-week-old nude mice, respectively, to establish xenograft leukemia models. Tumor formation was monitored for 28 days and measured every 3 days. Both the tumor size and tumor weight in mice injected with the FBXO42-KO JURKAT cells were significantly reduced compared with those in the mice injected with the control JURKAT cells (FIG. 13A-C). A similar result was obtained in the HSB2-induced xenograft mouse model (FIG. 13E-G). To evaluate whether the suppressive effect of FBXO42 on tumor formation is related to Notch signaling modulation, tumor tissues derived from different cells were isolated, and the expression of classical Notch target genes was detected. Notch target gene expression was reduced in the FBXO42-KO cells formed tumors (FIGS. 13D and 13H).

We established another mouse model via tail vein injection of leukemia cells to explore the effect of FBXO42 expression on leukemia cell invasion in vivo. GFP-labeled WT FBXO42 and FBXO42-KO JURKAT cells were injected into immune-deficient NSG mice via the tail vein, and leukemia progression was monitored weekly by measuring the GFP intensity through flow cytometry analysis (FIG. 13I). We observed that knocking out FBXO42 significantly decreased the leukemia burden and progression in peripheral blood (FIGS. 13J and 13K), as well as splenomegaly (FIGS. 13L and 13M). We found that leukemia cell infiltration in the spleen and abnormal spleen histology were attenuated in the FBXO42-KO group (FIGS. 13N and 13O). Moreover, bioluminescence imaging with luciferase-containing WT FBXO42 and FBXO42-KO JURKAT cells also confirmed the suppressive effect of FBXO42 on leukemia progression and mouse survival (FIG. 13P-R). As MLN4924 inhibited leukemia cell viability, we detected its effect in a JURKAT xenograft model. As evidenced by the tumor growth rate, pharmacological inhibition of FBXO42 activity by MLN4924 reduced the leukemia burden in vivo without inducing obvious toxicity (FIG. 13S-V).

Together, these data suggested that FBXO42 plays a key role in leukemia tumorigenesis both in vitro and in vivo and may be a potential drug target for the interference of Notch-related diseases, especially T-ALL.

The present invention is not limited to above embodiments. Any variation, modification, substitution, combination, and simplification without departing from the spirit and principle of the present invention belongs to equivalents of the present invention and is included within the scope of protection of the present invention.

1. A method for treating Notch signaling-dependent disease in the subject with a FBXO42 specific inhibitor, wherein the Notch signaling-dependent disease include activating mutations and/or amplification of Notch gene and/or Notch pathway activity, preferably, the Notch signaling-dependent disease is selected from leukemia, myeloma, lymphoma, breast cancer, liver cancer, head and neck squamous cell carcinoma (HNSCC), lung cancer and other cancers carrying the activating mutations and/or amplification of Notch gene and/or Notch pathway activity.

2. The method of claim 1, wherein the FBXO42 specific inhibitor is a polypeptide antagonist specifically against FBXO42, a polynucleotide specific to FBXO42, or a small molecule compound inhibitor specific to FBXO42.

3. The method of claim 2, wherein the polynucleotide is selected from siRNA, shRNA, guide RNA, miRNA, ASO.

4. The method of claim 3, wherein the guide RNA comprises:
1) a nucleotide sequence of SEQ ID NO:1 (cggcccttgtctgcaaacag);
2) a nucleotide sequence at least about 70%, about 80%, about 85%, about 90%, about 95%, about 99%, or more identity to SEQ ID NO:1; or
3) a nucleotide sequence with addition, deletion and/or substitution of one or more amino acids compared with SEQ ID NO: 1,
the polynucleotide specific to FBXO42 can bind to FBXO42 gene, preventing its translation.

5. The method of claim 2, wherein the polypeptide antagonist is an antibody against FBXO42, preventing ligands such as RBPJ from its binding, preferably, the antibody specifically binds to FBXO42.

6. The method of claim 1, wherein the Notch signaling-dependent disease is selected from leukemia, myeloma, lymphoma, breast cancer, liver cancer, head and neck squamous cell carcinoma (HNSCC), and lung cancer with Notch related mutations, more preferably, Notch related mutations comprise Notch1, Notch2 and/or Notch3 mutations.

7. The method of claim 6, wherein the leukemia is T-acute lymphoblastic leukemia or Chronic lymphocytic leukemia.

8. The method of claim 6, wherein the lymphoma is Hodgkin lymphoma, Burkitt lymphoma, Diffuse large B-cell lymphoma, Mantle cell lymphoma, Splenic marginal zone lymphoma, or Follicular lymphoma.

9. The method of claim 1, wherein the subject is non-human mammal or human.

10. The method of claim 1, wherein the disease is a metastatic cancer.

11. A method of screening medicines for treating Notch signaling-dependent disease using FBXO42 as the target, the method comprising: observing the effect of candidate medicine on the expression or activity level of FBXO42, if the candidate medicine can inhibit expression or activity level of FBXO42, then it indicates that the candidate medicine is a potential medicine for treating Notch signaling-dependent disease, preferably the Notch signaling-dependent disease include activating mutations and/or amplification of Notch gene and/or Notch pathway activity.

12. The method of claim 11, wherein the Notch signaling-dependent disease is selected from leukemia, myeloma, lymphoma, breast cancer, liver cancer, head and neck squamous cell carcinoma (HNSCC), lung cancer and other cancers carrying the activating mutations and/or amplification of Notch gene and/or Notch pathway activity; more preferably, the Notch signaling-dependent disease is selected from leukemia, myeloma, lymphoma, breast cancer, liver cancer, head and neck squamous cell carcinoma (HNSCC), and lung cancer with Notch related mutations, more preferably, Notch related mutations comprise Notch1, Notch2 and/or Notch3 mutations.

13. The method of claim 12, wherein leukemia is T-acute lymphoblastic leukemia or Chronic lymphocytic leukemia.

14. The method of claim 12, wherein the lymphoma is Hodgkin lymphoma, Burkitt lymphoma, Diffuse large B-cell lymphoma, Mantle cell lymphoma, Splenic marginal zone lymphoma, or Follicular lymphoma.
